(19) United States
(12) Patent Application Publication
(10) Pub. No.: US 2014/0114336 A1
(43) Pub. Date: Apr. 24, 2014

(54) MINIMALLY INVASIVE MICRO TISSUE DEBRIDERS HAVING TARGETED ROTOR POSITIONS

(71) Applicants: Gregory P. SCHMITZ, Los Gatos, CA (US); Juan Diego PEREA, Campbell, CA (US); Ming-Ting WU, Northridge, CA (US); Richard T. CHEN, Woodland Hills, CA (US); Arun VEERAMANI, Woodland Hills, CA (US)

(72) Inventors: Gregory P. SCHMITZ, Los Gatos, CA (US); Juan Diego PEREA, Campbell, CA (US); Ming-Ting WU, Northridge, CA (US); Richard T. CHEN, Woodland Hills, CA (US); Arun VEERAMANI, Woodland Hills, CA (US)

(21) Appl. No.: 13/659,734

(22) Filed: Oct. 24, 2012

(51) Int. Cl. A61B 17/32

(52) U.S. Cl. CPC A61B 17/320016
USPC 606/170

(57) ABSTRACT

A medical device for removing tissue from a subject is provided with a distal housing, an elongate member, a first rotatable member and first and second tissue shearing surfaces. The distal housing is configured with at least one tissue engaging opening. The elongate member is coupled to the distal housing and configured to introduce the distal housing to a target tissue site. The first rotatable member is located at least partially within the distal housing. The first and second tissue shearing surfaces are located and configured to cooperate with first and second sides of a first blade to shear tissue therebetween. The first rotatable member is configured to engage tissue from the target tissue site, rotate towards the first and second tissue shearing surfaces and inwardly to direct tissue from the target tissue site through the tissue engaging opening and into an interior portion of the distal housing.

### CROSS REFERENCE TO RELATED APPLICATIONS

This application is related to the following U.S. applications: application Ser. No. 13/535,197 filed Jun. 27, 2012; application Ser. No. 13/388,653 filed Apr. 16, 2012; application Ser. No. 13/289,994 filed Nov. 4, 2011; application Ser. No. 13/007,578 filed Jan. 14, 2011; application Ser. No. 12/491,220 filed Jun. 24, 2009; application Ser. No. 12/490,301 filed Jun. 23, 2009; application Ser. No. 12/490,295 filed Jun. 23, 2009; Provisional Application No. 61/710,608 filed Oct. 5, 2012; Provisional Application No. 61/408,558 filed Oct. 29, 2010; Provisional Application No. 61/234,989 filed Aug. 18, 2009; Provisional Application No. 61/075,007 filed Jun. 24, 2008; Provisional Application No. 61/075,006 filed Jun. 23, 2008; Provisional Application No. 61/164,864 filed Mar. 30, 2009; and Provisional Application No. 61/164,883 filed Mar. 30, 2009.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

Embodiments of the present disclosure relate to micro-scale and millimeter-scale tissue debridement devices that may, for example, be used to remove unwanted tissue or other material from selected locations within a body of a patient during a minimally invasive or other medical procedure, and in particular embodiments, multi-layer, multi-material electrochemical fabrication methods that are used to, in whole or in part, form such devices.

### BACKGROUND OF THE INVENTION

Debridement is the medical removal of necrotic, cancerous, damaged, infected or otherwise unwanted tissue. Some medical procedures include, or consist primarily of, the mechanical debridement of tissue from a subject. Rotary debrider devices have been used in such procedures for many years.

Some debrider devices with relatively large dimensions risk removing unintended tissue from the subject, or damaging the unintended tissue. There is a need for tissue removal devices which have small dimensions and improved functionality which allow them to more safely remove only the desired tissue from the patient. There is also a need for tissue removal devices which have small dimensions and improved functionality over existing products and procedures which allow them to more efficiently remove tissue from the patient.

Prior art tissue removal devices often remove tissue in large pieces, having dimensions well over 2 mm. The tissue pieces are removed through an aspiration lumen typically 3.5 to 5 mm in diameter. Since the tissue pieces being removed commonly have dimensions that are 1 to 2 lumen diameters in length, the tissue pieces can often clog the tissue removal lumen.

One portion of the body in which tissue can be removed to treat a variety of conditions is the spine area. Tissue removal devices for the spine are needed that can be produced with sufficiently small dimensions and/or that have increased performance over existing techniques. For example, a herniated disc or bulging disc can be treated by performing a discectomy, e.g. by removing all or part of the nucleus pulposus of the damaged disc. Such procedures may also involve a laminotomy or laminectomy wherein a portion or all of a lamina may be removed to allow access to the herniated disc. Artificial disc replacement (total or partial) is another example of a procedure which requires the removal of all or a portion of the disc, which is replaced with an artificial device or material.

Tissue removal devices are needed which can be produced with sufficient mechanical complexity and a small size so that they can both safely and more efficiently remove tissue from a subject, and/or remove tissue in a less invasive procedure and/or with less damage to adjacent tissue such that risks are lowered and recovery time is improved.

### SUMMARY OF THE DISCLOSURE

According to some aspects of the disclosure, a medical device for removing tissue from a subject is provided. One exemplary device includes a distal housing, an elongate member, a first rotatable member, and first and second tissue shearing surfaces. The distal housing is configured with at least one tissue engaging opening. The elongate member is coupled to the distal housing and is configured to introduce the distal housing to a target tissue site of the subject. The elongate member has a central longitudinal axis. The first rotatable member is located at least partially within the distal housing and is configured to rotate about a singular first axis. The first rotatable member comprises a first cutting blade which has a first side and a second side opposite the first side. The first tissue shearing surface is located and configured to cooperate with the first side of the first blade to shear tissue therebetween. The second tissue shearing surface is located and configured to cooperate with the second side of the first blade to shear tissue therebetween. The first rotatable member is configured to engage tissue from the target tissue site, rotate towards the first and second tissue shearing surfaces and inwardly to direct tissue from the target tissue site through the tissue engaging opening and into an interior portion of the distal housing.

In some embodiments, the first cutting blade comprises a disc-shaped portion having a series of teeth along an outer circumference of the blade. The disc-shaped portion may be configured to be perpendicular to the singular first axis. In some embodiments, at least one of the first and second tissue shearing surfaces is formed by a fixed portion of the distal housing.

In some embodiments, the first axis of the first rotatable member is coincident with the longitudinal axis of the elongate member. In other embodiments, the first axis of the first rotatable member intersects the longitudinal axis of the elongate member and is perpendicular therewith. In still other embodiments, the first axis of the first rotatable member intersects the longitudinal axis of the elongate member and forms an angle therewith of between 0 and 90 degrees.

In some embodiments, the first axis of the first rotatable member is offset from and parallel to the longitudinal axis of the elongate member and lies in a common plane therewith. In other embodiments, the first axis of the first rotatable member is offset from and perpendicular to the longitudinal axis of the elongate member and lies in a common plane therewith. In still other embodiments, the first axis of the first rotatable member is offset from the longitudinal axis of the elongate member, lies in a common plane and forms an angle therewith of between 0 and 90 degrees.

In some embodiments, the first axis of the first rotatable member is offset from and perpendicular to the longitudinal axis of the elongate member and lies in a different plane. At least one of the first and second tissue shearing surfaces may be formed by a second rotatable member located at least partially within the distal housing and configured to rotate about a singular second axis parallel to and offset from the first axis. The second rotatable member may be configured to rotate in a direction opposite of a direction of rotation of the first rotatable member. The second rotatable member may comprise a second disc-shaped blade having a series of teeth along an outer circumference of the blade. The second rotatable member may comprise a third disc-shaped blade having a series of teeth along an outer circumference of the blade. The three blades may be positioned such that they are interdigitated with one another.

In some embodiments, the first axis of the first rotatable member is offset from the longitudinal axis of the elongate member, lies in a different plane and forms an angle therewith of between 0 and 90 degrees. At least one of the first and second tissue shearing surfaces may be formed by a second rotatable member located at least partially within the distal housing and configured to rotate about a singular second axis parallel to and offset from the first axis. The second rotatable member may be configured to rotate in a direction opposite of a direction of rotation of the first rotatable member. The second rotatable member may comprise a second disc-shaped blade having a series of teeth along an outer circumference of the blade. The second rotatable member may comprise a third disc-shaped blade having a series of teeth along an outer circumference of the blade. The three blades may be positioned such that they are interdigitated with one another.

In some embodiments, the first axis of the first rotatable member is perpendicular to the longitudinal axis of the elongate member and is configured to articulate with respect thereto. The first axis may pivot about an articulation axis that is parallel thereto, or it may pivot about an articulation axis that is perpendicular thereto.

In some embodiments, the elongate member comprises a distal portion that is oriented at an angle with respect to a more proximal portion of the elongate member such that the central longitudinal axis has an inflection point between the distal portion and more proximal portion. In some of these embodiments, a distal portion of the central longitudinal axis and a more proximal portion of the central longitudinal axis may lie in a common plane that is coincident with or generally parallel to the first axis of the first rotatable member. In others of these embodiments, a distal portion of the central longitudinal axis and a more proximal portion of the central longitudinal axis may lie in a common plane that is generally perpendicular to the first axis of the first rotatable member.

In some embodiments, the elongate member comprises a generally rigid, curved distal portion and a generally straight more proximal portion. A curved, distal portion of the central longitudinal axis may lie in a plane that is coincident with or generally parallel to the first axis of the first rotatable member. A curved distal portion of the central longitudinal axis may lie in a plane that is generally perpendicular to the first axis of the first rotatable member.

Other aspects of the disclosure will be understood by those of skill in the art upon review of the teachings herein. Other aspects of the disclosure may involve combinations of the above noted aspects of the disclosure. These other aspects of the disclosure may provide various combinations of the aspects presented above as well as provide other configurations, structures, functional relationships, and processes that have not been specifically set forth above.

### BRIEF DESCRIPTION OF THE DRAWINGS

FIGS. 1-3 illustrate an exemplary embodiment of a working end of a tissue removal device.

FIGS. 4A-4G illustrate exemplary embodiments of drive mechanisms which can power the drive trains in the working end of tissue removal devices.

FIGS. 5A-5C show another exemplary embodiment of a tissue removal device.

FIGS. 6A-6C show an exemplary cutter head assembly 5332 that may be used with debriding device 5310, shown in FIGS. 5A-5C.

FIGS. 7A-7F show details of an exemplary rotor housing assembly 5420′.

FIGS. 8A-8T schematically show various rotor orientations enabled by the present disclosure.

FIGS. 9A-9H show the distal end of an exemplary embodiment of a side mount tissue shredder.

FIGS. 10A-10C show the distal end of an exemplary embodiment of an angled tissue shredder.

FIG. 10D is a cross-sectional view taken along line 10D-10D in FIG. 10C.

FIG. 10E is a cross-sectional view taken along line 10E-10E in FIG. 10C.

FIG. 10F is a cross-sectional view taken along line 10E-10F in FIG. 10C.

FIGS. 11A-11B show the distal end of another exemplary embodiment of an angled tissue shredder

FIGS. 12A-12B show the distal end of yet another exemplary embodiment of an articulating tissue shredder.

### DETAILED DESCRIPTION

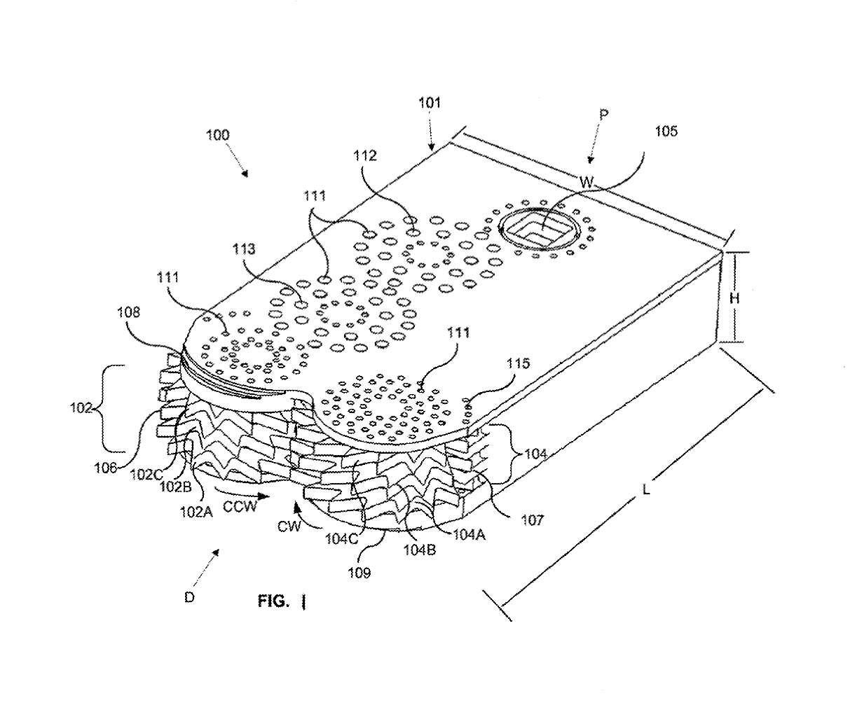

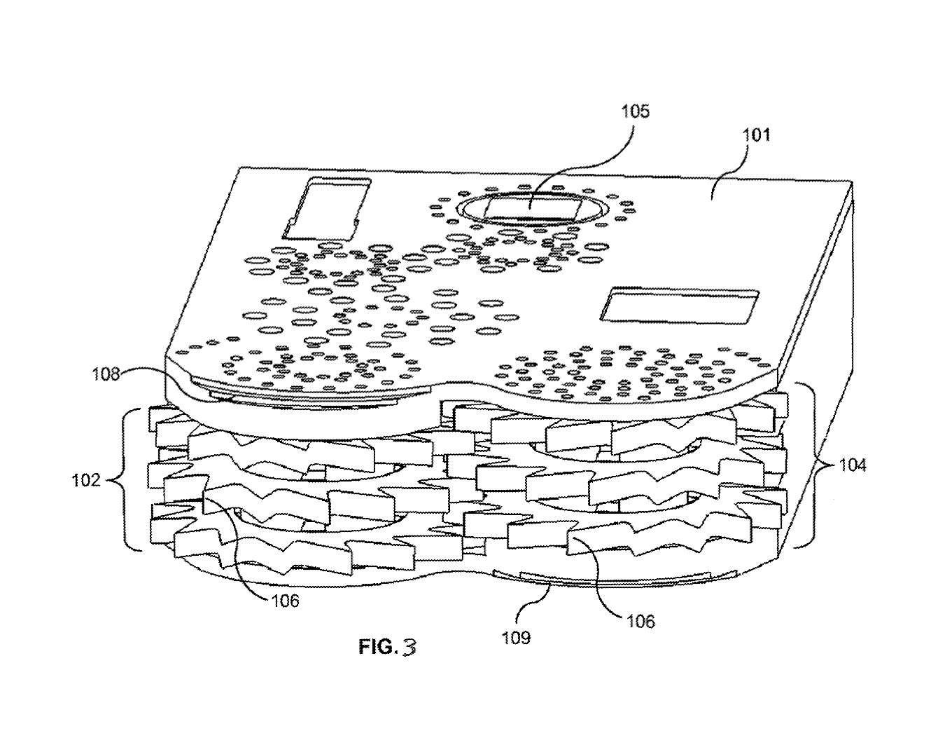

FIGS. 1-3 illustrate an exemplary embodiment of a working end of a tissue removal device, which can be fabricated wholly or in part by electrochemical fabrication techniques, such as those described or referenced herein. Tissue removal device working end 100 has a distal region “D” and proximal region “P,” and includes housing 101 and blade stacks 102 and 104. Blade stacks 102 and 104 include a plurality of blades 102A-102C and 104A-104C, respectively. Three blades are shown in each stack, although the blade stacks can have one or more blades. Each of the blades includes a plurality of teeth 106 (see FIG. 3), some of which are shown projecting from housing 101 and configured to engage and process tissue. Processing tissue as used herein includes any of cutting tissue, shredding tissue, capturing tissue, any other manipulation of tissue as described herein, or any combination thereof. The working end of the device generally has a length L, height H, and width W. Housing 101 can have a variety of shapes or configurations, including a generally cylindrical shape.

In this embodiment both blade stacks are configured to rotate. The blades in blade stack 102 are configured to rotate in a direction opposite that of the blades in blade stack 104, as designated by the counterclockwise “CCW” and clockwise “CW” directions in FIG. 1. The oppositely rotating blades direct material, such as tissue, into an interior region of housing 101 (described in more detail below). In some embodiments, the blades can be made to be rotated in directions opposite to those indicated, e.g. to disengage from tissue if a jam occurs or to cause the device to be pulled distally into a body of tissue when given appropriate back side teeth configurations.

Housing 101 also includes a drive mechanism coupler 105, shown as a square hole or bore, which couples a drive train disposed in the housing to a drive mechanism disposed external to the housing. The drive mechanism, described in more detail below, drives the rotation of the drive train, which drives the rotation of the blades. The drive train disposed in the housing can also be considered part of the drive mechanism when viewed from the perspective of the blades. Drive mechanism coupler 105 translates a rotational force applied to the coupler by the drive mechanism (not shown) to the drive train disposed within housing 101.

FIG. 1 also shows release holes 111-115 which allow for removal of sacrificed material during formation of the working end.

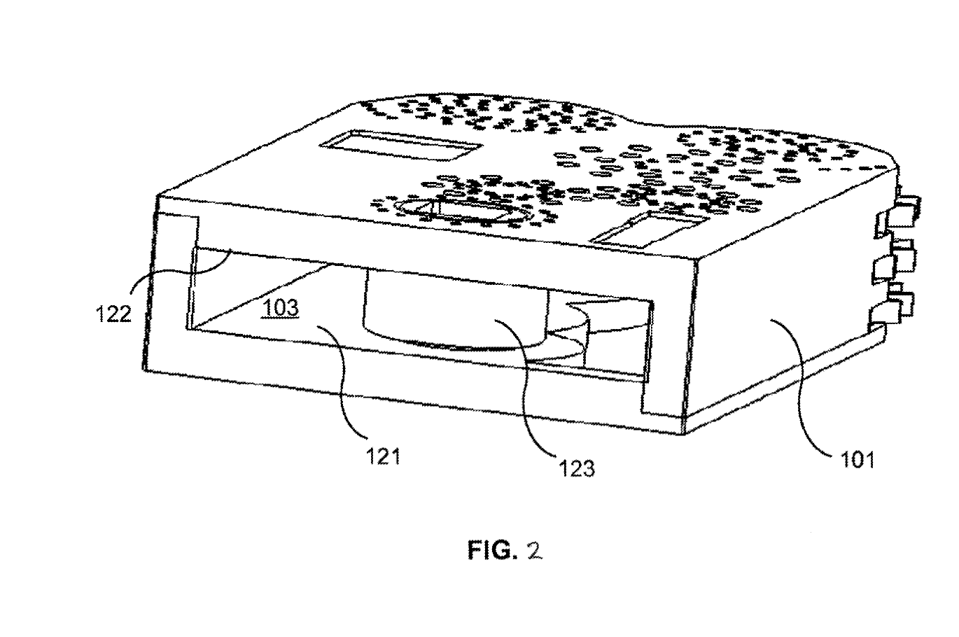

FIG. 2 shows a perspective view of the proximal end of tissue removal device working end 100. Material directed into housing 101 by the rotating blades is directed into chamber 103, wherein it can be stored temporarily or directed further proximally, as described below. A first gear train cover 121 provides for a first surface of chamber 103, while a second gear train cover 122 provides a second surface of chamber 103. FIG. 2 also shows drive mechanism coupler cover 123.

In some embodiments in which the working end 100 includes a storage chamber, the chamber may remain open while in other embodiments it may be closed while in still other embodiments it may include a filter that only allows passage of items of a sufficiently small size to exit.

FIG. 3 shows a perspective view of the distal end of the working end 100. In this embodiment the blades in stack 102 are interdigitated with the blades in stack 104 (i.e. the blade ends are offset vertically along dimension H and have maximum radial extensions that overlap laterally along the width dimension W. The blades can be formed to be interdigitated by, e.g. if formed using a multi-layer, multi-material electrochemical fabrication technique, forming each blade in stack 102 in a different layer than each blade in stack 104. If during formation portions of separately moveable blade components overlap laterally, the overlapping blades should not just be formed on different layers but should be formed such an intermediate layer defines a vertical gap between them. For example, the bottom blade in stack 102 is shown formed in a layer beneath the layer in which the bottom blade in stack 104 is formed.

When manufacturing tissue removal devices of the various embodiments set forth herein using a multi-layer multi-material electrochemical fabrication process, it is generally beneficial if not necessary to maintain horizontal spacing of component features and widths of component dimensions remain above the minimum feature size. It is important that vertical gaps of appropriate size be formed between separately movable components that overlap in X-Y space (assuming the layers during formation are being stacked along the Z axis) so that they do not inadvertently bond together and to ensure that adequate pathways are provided to allow etching of sacrificial material to occur. For example, it is generally important that gaps exist between a gear element (e.g. a tooth) in a first gear tier and a second gear tier so that the overlapping teeth of adjacent gears do not bond together. It is also generally important to form gaps between components that move relative to one another (e.g., gears and gear covers, between blades and housing, etc.). In some embodiments the gaps formed between moving layers is between about 2 um and about 8 um.

In some embodiments, it is desired to define a shearing thickness as the gap between elements has they move past one another. Such gaps may be defined by layer thickness increments or multiples of such increments or by the intralayer spacing of elements as they move past one another. In some embodiments, shearing thickness of blades passing blades or blades moving past interdigitated fingers, or the like may be optimally set in the range of 2-100 microns or some other amount depending on the viscosity or other parameters of the materials being encountered and what the interaction is to be (e.g. tearing, shredding, transporting, or the like). For example for shredding or tearing tissue, the gap may be in the range of 2-10 microns, or in some embodiments in the range of 4-6 microns.

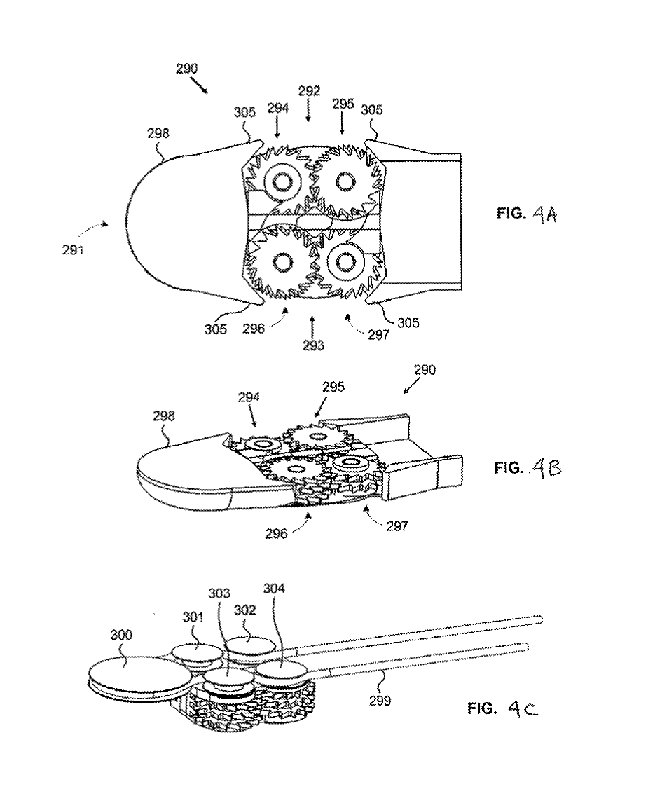

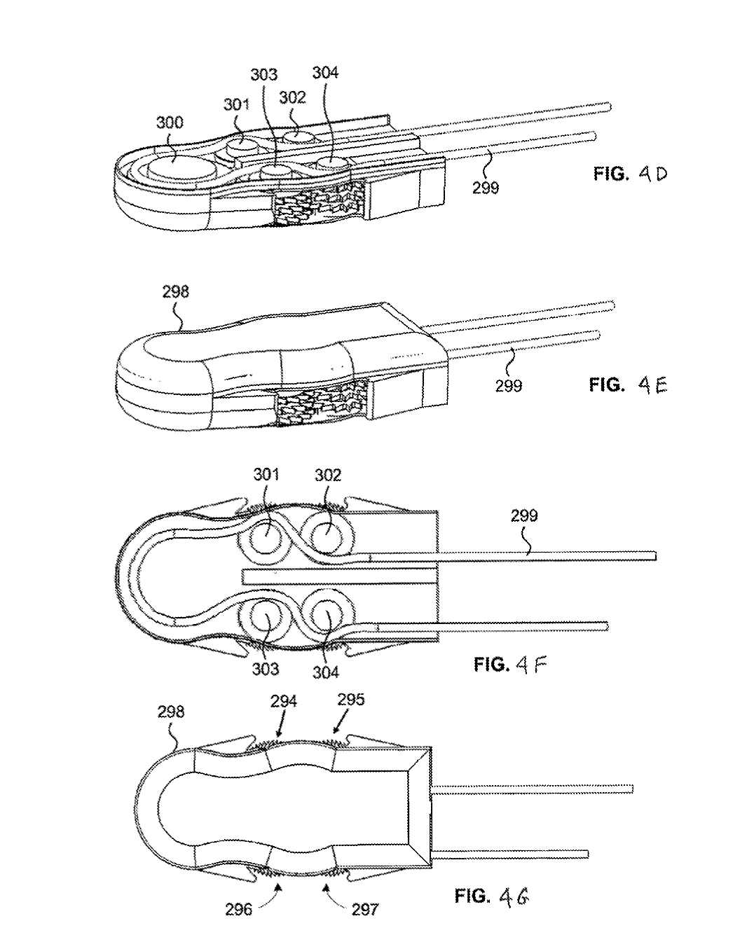

FIGS. 4A-4G illustrate an example a of a side tissue removal working end. FIG. 4A is a top sectional view with a top portion of the housing removed, which shows working end 290 comprising housing 298 and four tissue removal elements 294-297, which are shown as blade stacks. Blade stacks 294 and 295 process tissue along one side of the housing by directing tissue in the direction of arrow 292. Blade stacks 296 and 297 process tissue along a second side of the housing by directing tissue in the direction of arrow 293. As shown in FIGS. 4A-B, blade stacks 294 and 297 each have two blades, while blade stacks 295 and 296 each have three blades. FIG. 4C shows a perspective view without housing 298 illustrating the drive mechanism for the side tissue removal device 290. The drive mechanism includes belt 299, distal pulley 300, and side pulleys 301-304. The side pulleys are coupled to the blade stacks and rotation of the side pulleys rotates the blade stacks. The belt is disposed through side pulleys 301 and 302 and around distal pulley 300 before returning through side pulleys 303 and 304. Actuating of belt 299 therefore activates all four blade stacks. In some embodiments the belt is a nitinol wire, but can be any other suitable material. FIG. 4D is a view with the top portion of the housing removed to show the internal drive mechanism. FIG. 4E shows the same view with the top on the housing. FIGS. 4F and 4G show top views of the working end shown in FIGS. 4D and 4E, respectively. Vacuum, irrigation, or a combination of the two may be used to send extracted tissue from the interior of the working end, proximally to a storage reservoir (e.g. within the working end or located outside the body of the patient on which a procedure is being performed).

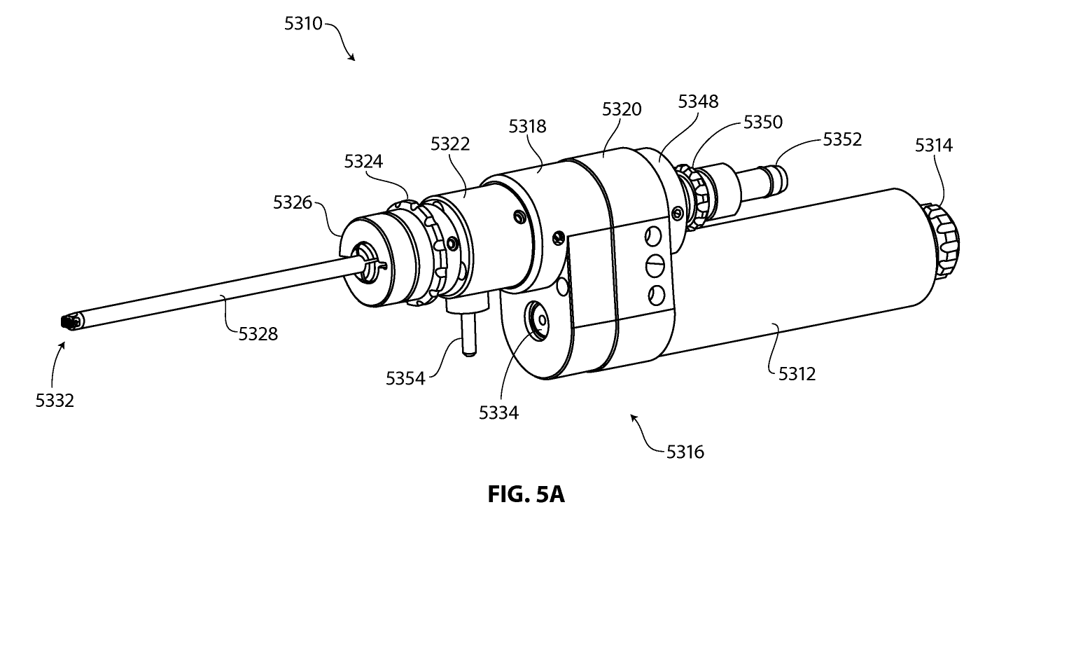

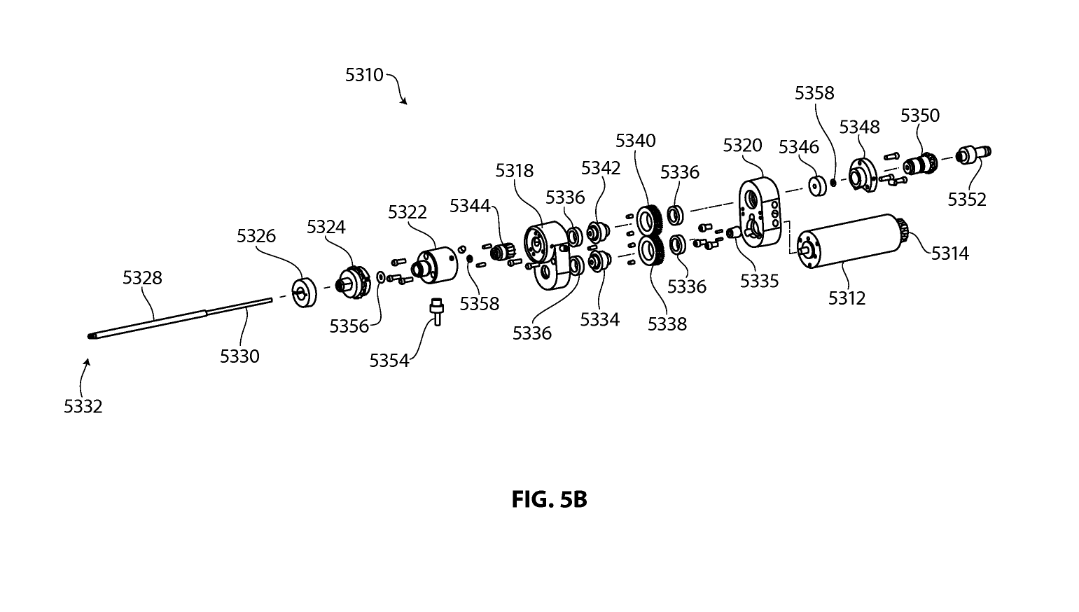

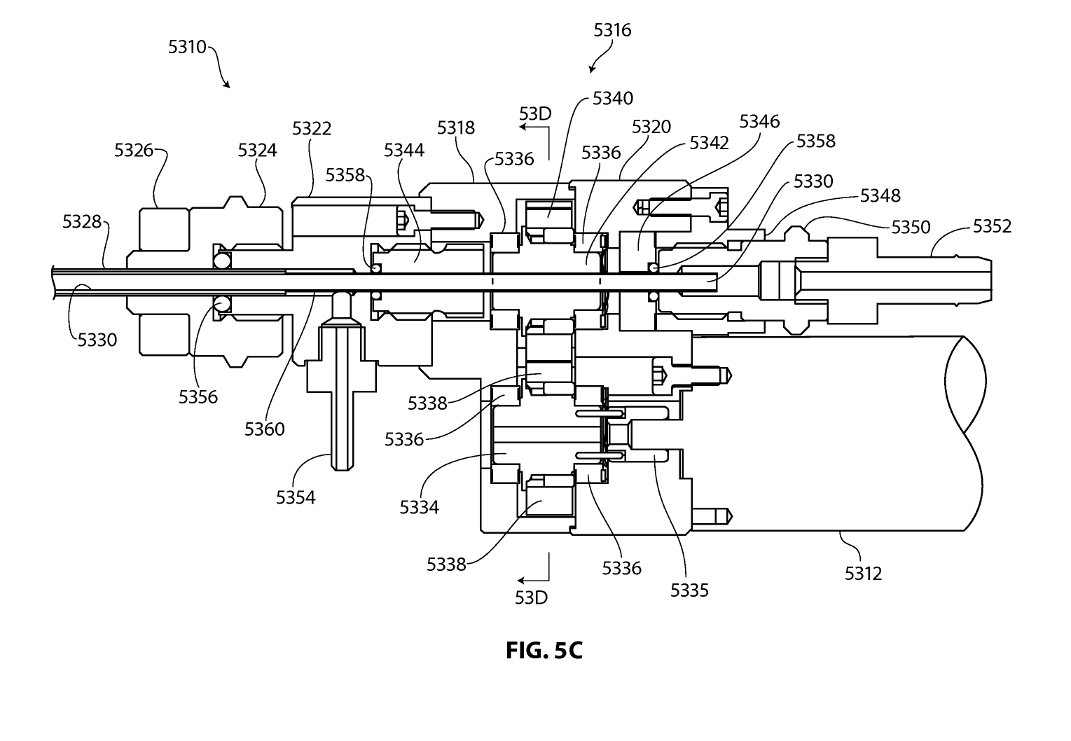

FIGS. 5A-5C show another exemplary embodiment of a tissue removal device. Device 5310 may employ any of the cutting heads described herein, or other suitable cutting heads. In some embodiments, a double rotor shredding head is employed at the distal end of device 5310 to selectively debride tissue down to the cellular level.

In this exemplary embodiment, handheld device 5310 includes a stepper motor 5312 at its proximal end. In other embodiments, other types of electric, pneumatic or hydraulic motors, servos, or other prime movers may be used. The proximal end of motor 5312 may be provided with a manually turnable thumbwheel 5314, as shown. In this embodiment, the distal output end of motor 5312 is provided with a housing 5316, which is made up of a front cover 5318 and a rear cover 5320. Located distally from housing 5316 are an outer shaft housing 5322, an outer shaft lock seal 5324, and a support clamp 5326. A non-rotating, outer support tube 5328 extends from within the proximal end of device 5310 towards the distal end of the device. Within support tube 5328, a rotating drive tube 5330 (best seen in FIGS. 5B and 5C) also extends from within the proximal end of device 5310 towards the distal end of the device. The support tube 5328 and inner drive tube 5330 may collectively be referred to as an introducer. A cutter head assembly 5332, subsequently described in detail, is attached to the distal end of support tube 5328.

As best seen in FIG. 5B, other components of device 5310 include motor shaft drive axle 5334, motor dog 5335, four bearings 5336, drive gear 5338, driven gear 5340, inner drive shaft axle 5342, inner shaft lock seal 5344, vacuum gland disk 5346, vacuum seal lock housing 5348, vacuum seal lock 5350, vacuum hose barb 5352, irrigation fluid hose barb 5354, outer tube o-ring 5356, and two vacuum gland o-rings 5358. Various other pins, dowels, fasteners, set screws, ball detents, shims and wave disc springs are shown in the figures without reference numerals. As will be appreciated by those skilled in this art, these non-referenced components serve to align, retain and ensure the proper functioning of the other components of exemplary device 5310.

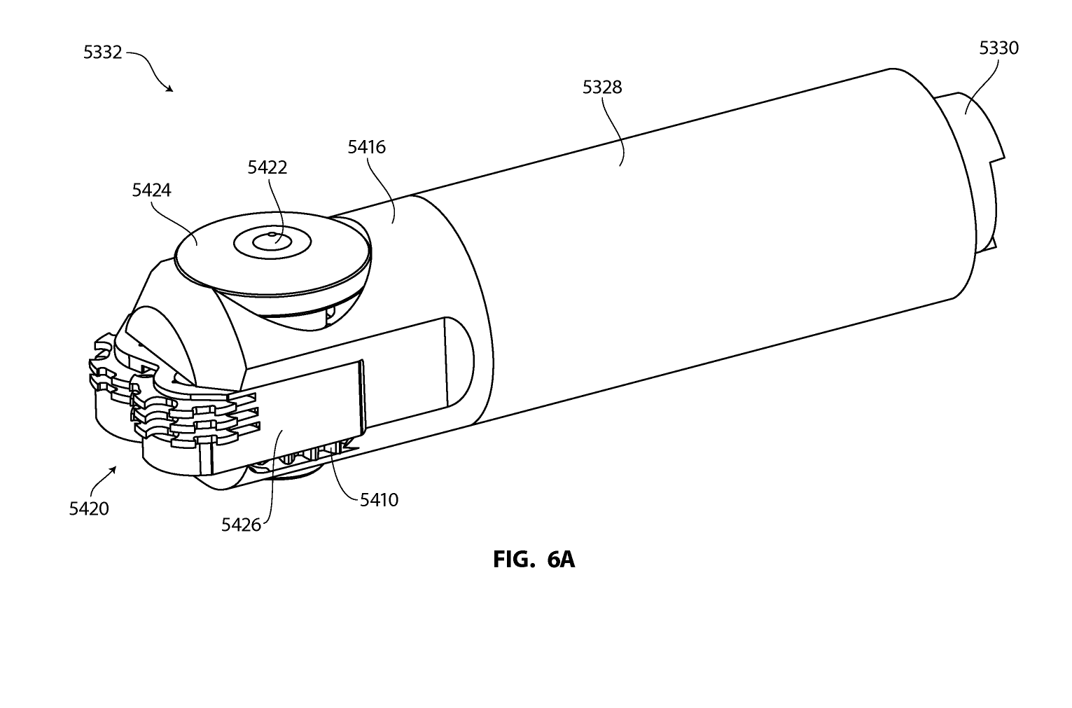

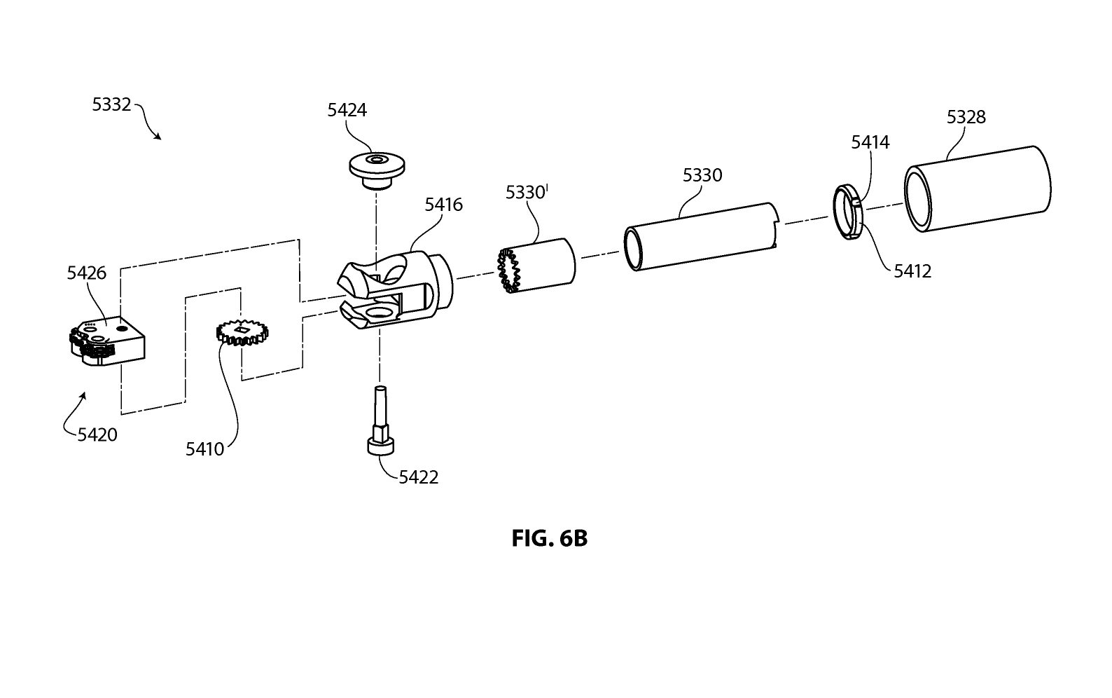

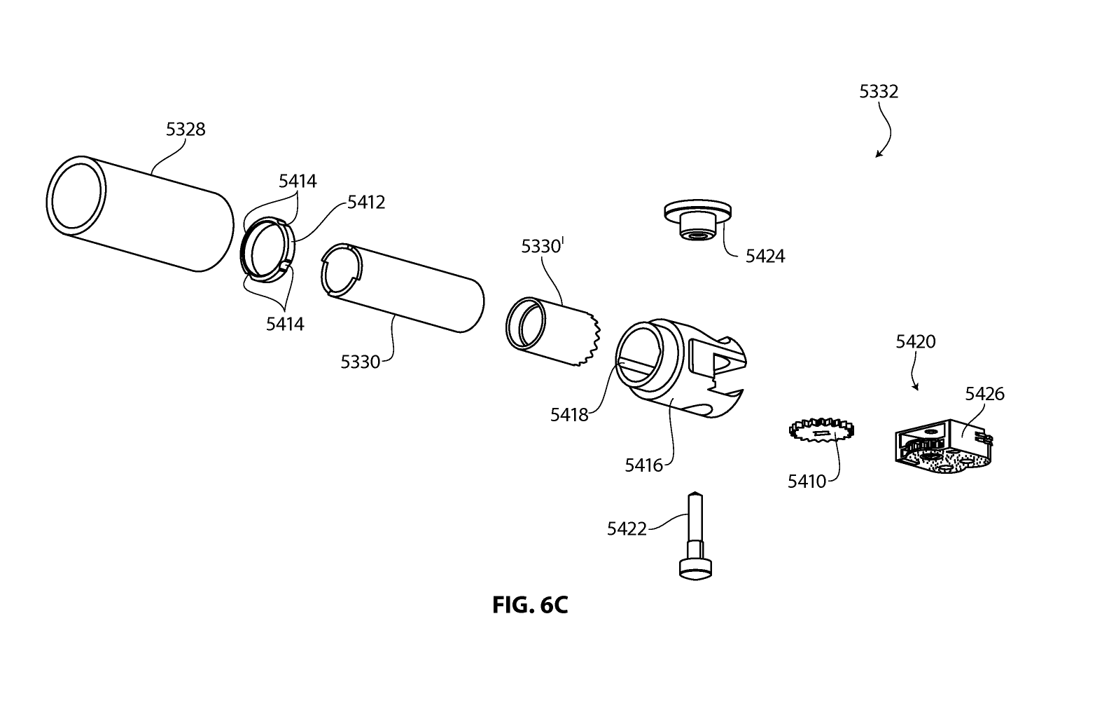

The two rotors of cutter head assembly 5332 located at the distal end of device 5310 are driven by motor 5312 through drive tube 5330 and other drive components of device 5310, as will now be described in more detail. As best seen in FIGS. 5B and 5C, a motor dog 5335 is attached to the output shaft of motor 5312. Motor dog 5335 is coupled to motor shaft drive axle 5334, which is rotatably mounted in housing 5316 with two bearings 5336. Drive gear 5338 is rigidly fixed to motor shaft drive axle 5334, and drives driven gear 5340. Driven gear 5340 is rigidly fixed to inner drive shaft axle 5342, which is rotatably mounted in housing 5316 with two bearings 5336. Inner rotating drive tube 5330 passes through the center of inner drive shaft axle 5342 and is rotatably fixed thereto. Drive tube 5330 extends from the proximal end of device 5310 to the distal end of the device through the non-rotating outer support tube 5328. The distal end of drive tube 5330 (or a separate tube 5330′ attached thereto) is provided with crown teeth around its periphery, as shown in FIGS. 6B and 6C, for meshing with drive gear 5410. As drive tube 5330 is rotated about a longitudinal axis of device 5310 by motor 5312 through the above-described drive train components, it drives drive gear 5410 about an axis that is perpendicular to the longitudinal axis, as can be appreciated by viewing FIG. 6. Drive gear 5410 in turn drives other components of the cutter head assembly, and as is subsequently described in more detail.

In some embodiments motor 5312 is provided with feedback control for rotational velocity and torque. These two parameters can be used for controlling and monitoring changes in rotational velocity and the torque load. For measuring rotational velocity, an encoder may be located at one or more of the cutter rotors, at the drive motor, or at another location along the drive train between the drive motor and cutter rotors. In some embodiments, the encoder is located at or close to the rotors to avoid backlash associated with the drive train, thereby making the velocity monitoring more responsive and accurate. Encoder technologies that may be used include optical, resistive, capacitive and/or inductive measurement. To sense torque load, one or more strain gages may be located at the cutter rotors, at the drive motor, or at another location along the drive train between the drive motor and cutter rotors. Torque load may also be sensed by monitoring the current being drawn by the motor. By sensing changes in velocity and/or torque, a controller associated with device 5310 can determine that the cutter rotors are passing from one tissue type to another and take appropriate action. For example, the controller can sense when the cutter elements are passing from soft to hard tissue, from hard to medium density tissue, or from a cutting state to non-cutting state. In response to these changes, the controller and/or device 5310 can provide audio, visual and/or tactile feedback to the surgeon. In some embodiments, the controller can change the velocity, direction or stop cutter rotors from rotating in response to velocity and/or torque feedback. In one embodiment of the invention, a typical cutting rotor speed is on the order of 100 to 20,000 rotations per minute, and a typical torque load is on the order of 0.25 to 150 mN-meter. Other sensors, such as a pressure sensor or strain sensor located at the distal tip of device 5310, may also be utilized to provide feedback that tissue cutting elements are moving from one tissue type to another. In some embodiments, an impendence sensor may be located at the distal tip of the device, to sense different tissue types or conditions, and provide corresponding feedback for tissue cutting control when the tissue being cut by the cutter head changes. Such a pressure sensor feedback control arrangement can be used with types of cutting devices other than those disclosed herein.

Referring now to FIG. 5C, irrigation fluid hose barb 5354 is provided on the lower side of outer shaft housing 5322 of exemplary device 5310. Hose barb 5354, or a similar fluid line coupling, may be connected to a supply of irrigation fluid. The lumen of hose barb 5354 is in fluid communication with an internal irrigation fluid cavity 5360. Fluid cavity 5360 surrounds internal drive tube 5330, and is bounded on its proximal end by o-ring seal 5358 around drive tube 5330. Fluid cavity 5360 is bounded on its distal end by o-ring seal 5356 around outer support tube 5328. This arrangement allows drive tube 5330 to rotate, but constrains irrigation fluid delivered from hose barb 5354 to travel only through the annular space defined by the outer surface of drive tube 5330 and the inner surface of support tube 5328. Irrigation fluid may thus flow distally through the annular space to the distal end of device 5310.

As shown in FIG. 6B, one or more drive aligner rings 5412 may be provided between outer support tube 5328 and inner drive tube 5330 along their lengths to support drive tube 5330 as it rotates. In order to allow the flow of irrigation fluid between the tubes 5328 and 5330, rings 5412 may be provided with one or more channels 5414 as shown. When the distal flow of irrigation fluid reaches the cutter head assembly 5332, it continues to flow distally into lug 5416. To enable the fluid flow, lug 5416 is provided with fluid channels 5418 located along the outer walls of its central bore, as best seen in FIG. 6C. In this embodiments, irrigation fluid passes distally between inner drive tube 5330 and lug 5416 through channels 5418 (only one channel shown in FIG. 6C). Irrigation fluid flowing distally through channels 5418 may be directed toward the outside portions of cutting elements. In this embodiment, the outside portions of cutting elements are rotating distally, away from the fluid flow, while the inside portions of cutting elements are rotating proximally, toward the center of lug 5416 and drive tube 5330.

In some embodiments, the irrigation fluid serves multiple functions. The irrigation fluid can serve to lubricate the cutting elements, drive gears, journal bearings and other components as the parts rotate. The irrigation fluid can also serve to cool the cutting elements and/or the tissue being cut, absorbing heat and carrying it away as the irrigation fluid is removed from the patient. The fluid can serve to flush tissue particles from the moving parts to prevent them from becoming clogged. The fluid can also serve to carry away the tissue portions being cut and remove them from the target tissue site. In some embodiments, the irrigation fluid is discharged from the cutting device and may be removed from the target tissue site with other, traditional aspiration means. With the current exemplary cutting device 5310, however, the irrigation fluid and/or other bodily fluids may be removed from the target tissue site by the cutting device 5310, as will now be described in detail.

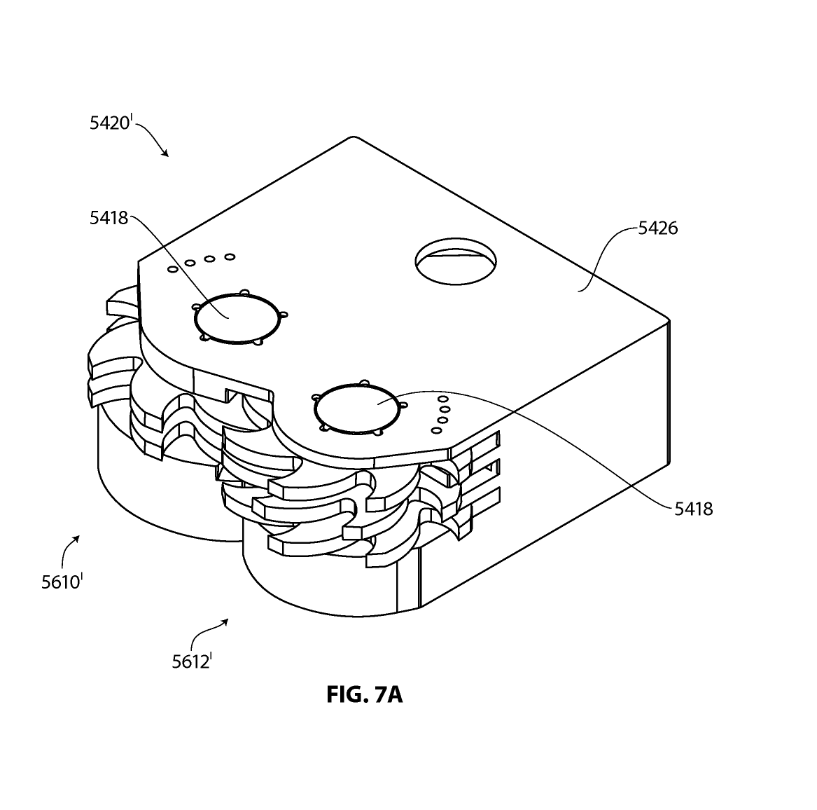

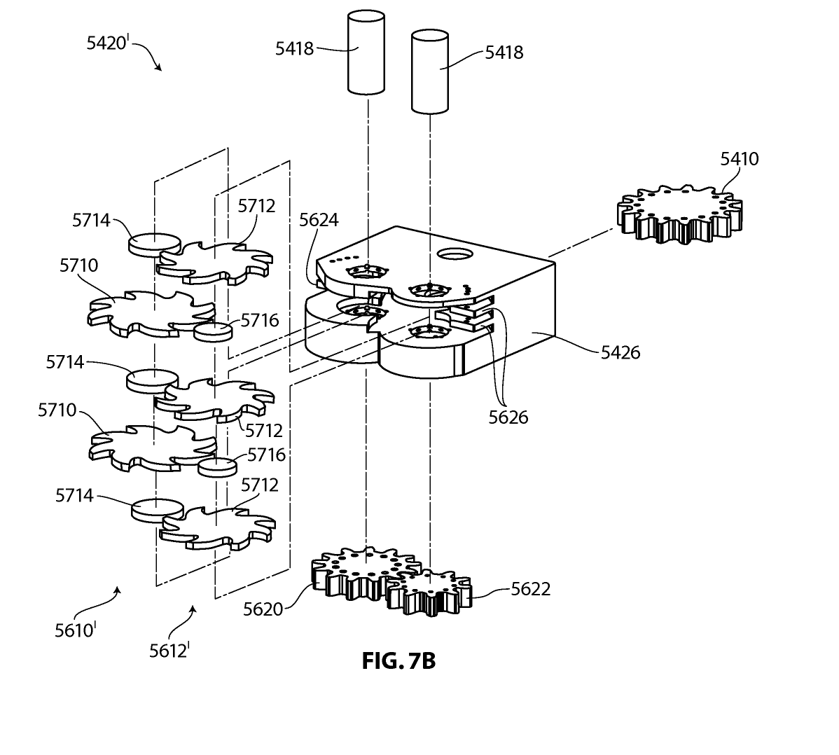

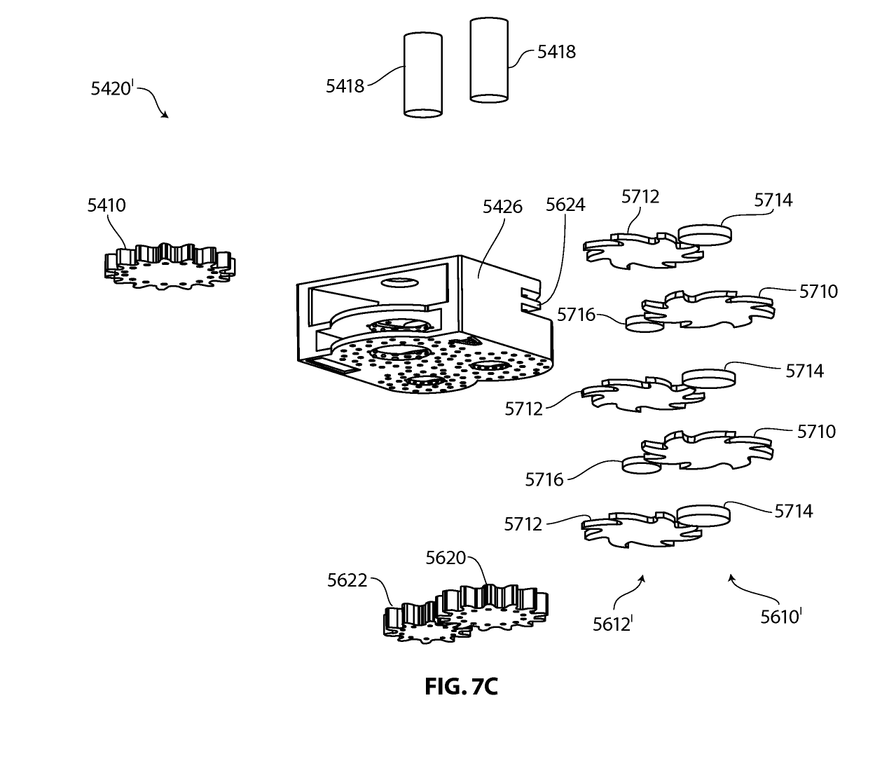

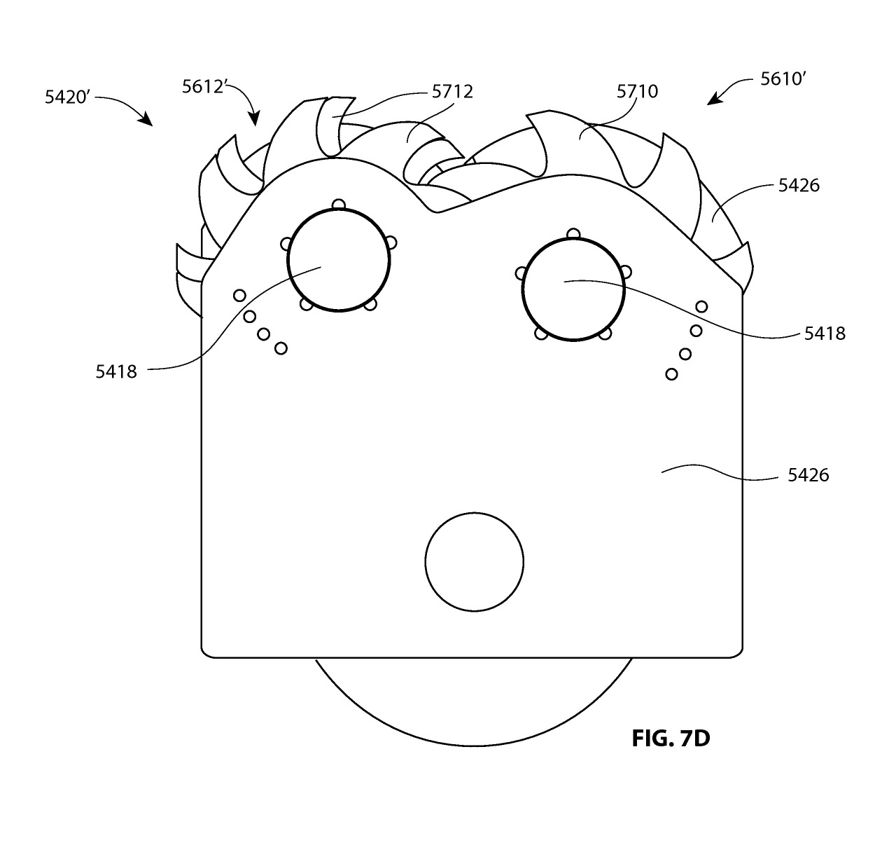

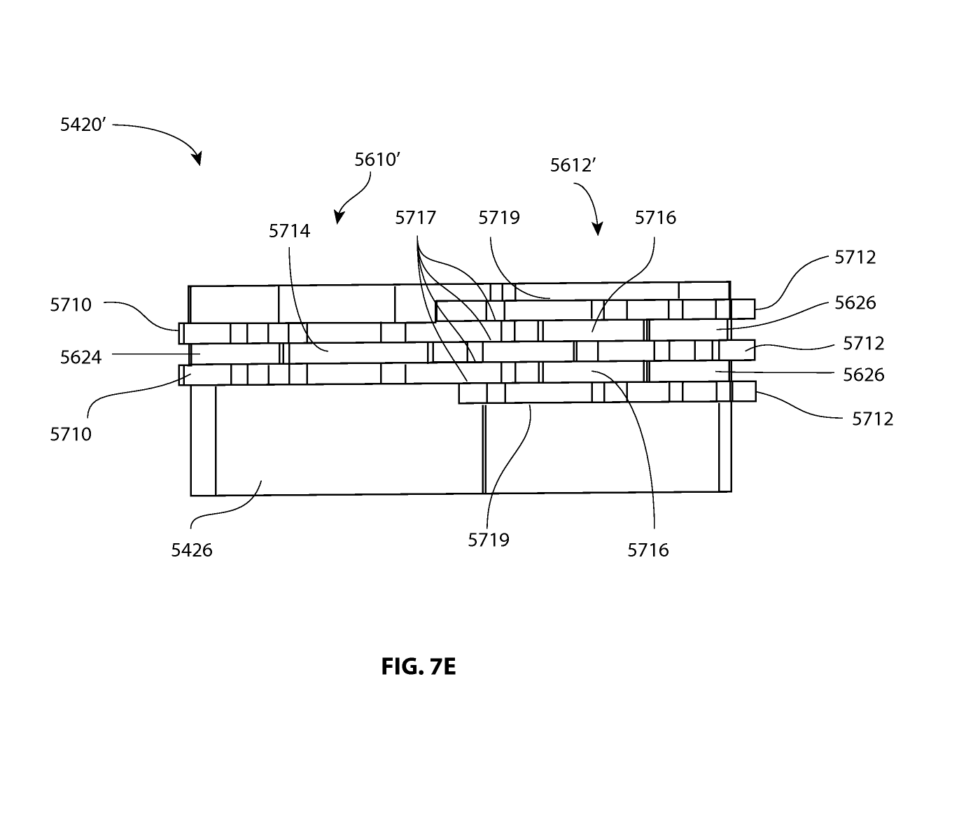

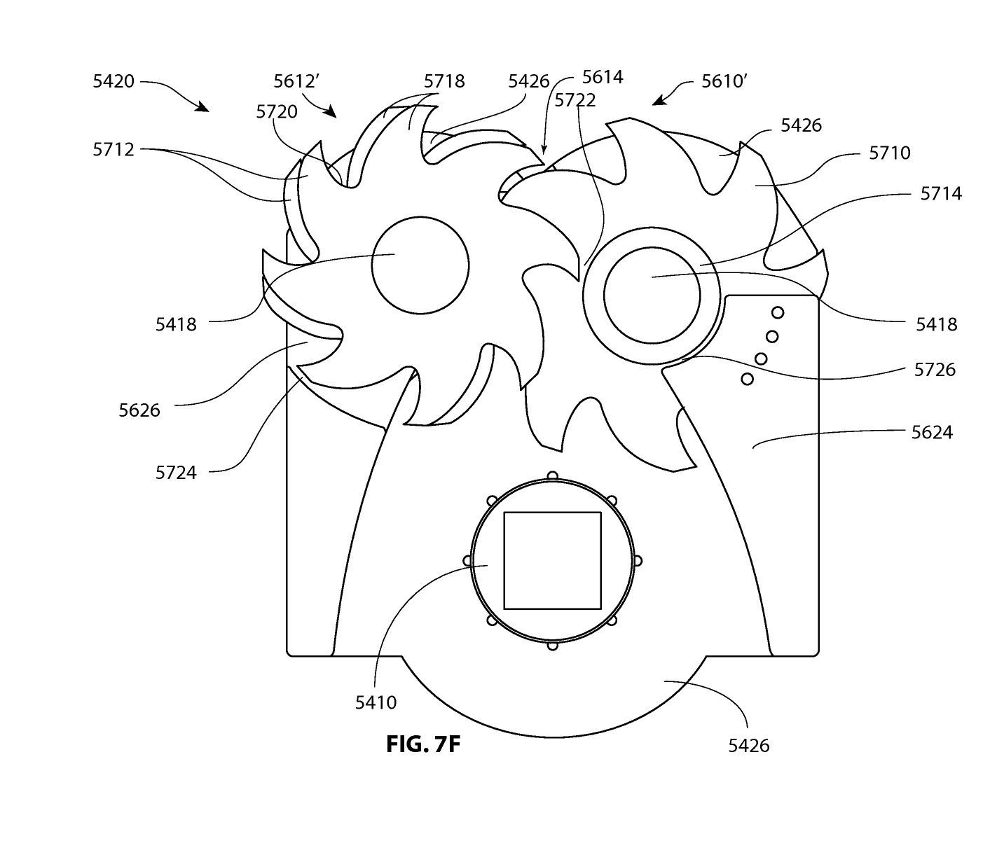

As previously described, irrigation fluid may be delivered to cutting elements and/or a target tissue site through device 5310. Exemplary device 5310 is also constructed to remove the irrigation fluid and tissue portions cut from the target tissue site through the shaft of device 5310. As can be appreciated by viewing FIG. 7F, the two interleaving stacks of cutting elements, also referred to as rotors 5610 and 5612, have an overlapping section 5614 in the center of cutter head assembly 5332. The two rotors 5610 and 5612 may be rotated in opposite directions such that each rotor engages target tissue and pulls it towards the central overlapping section 5614. In overlapping section 5614, the tissue is shredded into small pieces by the interdigitated cutting elements, as is subsequently described in more detail. The small tissue portions are generally propelled in a proximal direction by rotors 5610 and 5612, away from the target tissue site and into the cutter head assembly 5332. As can be appreciated by viewing FIG. 7F, the shredded tissue portions emerge from rotors 5610 and 5612 substantially along the central axis of lug 5416 (and therefore also the central axis of drive tube 5330. With sufficient irrigation fluid being supplied to the tissue cutting area, and sufficient aspiration being provided from the proximal end of the device, irrigation fluid around rotors 5610 and 5612 carries the cut tissue particles proximally down the center of drive tube 5330. As shown in FIG. 5C, the proximal end of drive tube 5330 is in fluid communication with hose barb 5352 located at the proximal end of device 5310. A traditional aspiration device or other suction source may be attached to device 5310 through hose barb 5352 or other suitable fluid coupling to collect the spent irrigation fluid and cut tissue portions.

In some embodiments, the cut tissues portions emerging from hose barb 5352 may be collected for testing. The tissue portions may be separated from the irrigation fluid, such as by centrifugal force, settling and/or filtering. The tissue portions may be measured to precisely determine the mass and/or volume of tissue removed. The pathology of some or all of the tissue portions may also be determined. In some embodiments, the above testing may be performed during a surgical procedure so that results of the testing may be used to affect additional stages of the procedure.

According to aspects of the invention, the inside diameter of drive tube 5330 may be much larger than the maximum dimension of the tissue portions traveling through it. In some embodiments, the maximum tissue dimension is less than about 2 mm across. In one exemplary embodiment, the inside diameter of drive tube 5330 is about 3 mm, the outside diameter of the support tube 5328 is about 5.6 mm, and the maximum dimension of the tissue portions is about 150 microns. In another exemplary embodiment, the inside diameter of drive tube 5330 is about 1.5 mm, the outside diameter of the support tube 5328 is about 2.8 mm, and the maximum dimension of the tissue portions is about 75 microns. In other embodiments, the inside diameter of drive tube 5330 is between about 3 mm and about 6 mm. In some embodiments, the maximum dimension of the tissue portions is at least one order of magnitude less than a diameter of the tissue removal lumen. In other embodiments, the maximum dimension of the tissue portions is at least twenty times less than a diameter of the tissue removal lumen. In some embodiments, the maximum dimension of the tissue portions is less than about 100 microns. In other embodiments, the maximum dimension of the tissue portions is about 2 microns.

Referring now to FIGS. 6A-6C, an exemplary cutter head assembly 5332 is described in more detail. Cutter head assembly 5332 may be used with debriding device 5310, shown in FIGS. 6A-6C. As best seen in FIG. 6B, cutter head assembly 5332 includes lug 5416, drive gear 5410, rotor housing assembly 5420, aligner pin 5422, and aligner cap 5424. Lug 5416 is provided with a cutout on its distal end for receiving rotor housing assembly 5420. Beneath the rotor housing cutout, lug 5416 has a circular recess for receiving drive gear 5410. A bore is provided in the bottom of lug 5416 for receiving the head of aligner pin 5422. When cutter head 5332 is assembled, the shank of aligner pin 5422 passes through the bore of lug 5416, through a square aperture in the center of drive gear 5410, through a bore in the proximal end of rotor housing assembly 5420, and into a large diameter bore through the top of lug 5416. Aligner cap 5424 is received with the large diameter bore in the top of lug 5416, and is fastened to aligner pin 5422 by a press fit, weld, threads, a separate fastener, or other suitable means. In this assembled arrangement, pin 5422 and cap 5424 retain rotor housing 5426 from moving longitudinally relative to the central axis of the instrument, and rotor housing 5426 and drive gear 5410 retain pin 5422 and cap 5424 from moving radially relative to the central axis of the instrument. Pin 5422 and cap 5424 spin together as a unit relative to lug 5416, and serve to align drive gear with the distal end of drive tube 5330′, as previously described. Pin 5422 also serves to transmit torque from drive gear 5410 to gear 5616, which resides inside the rotor housing directly above drive gear 5410. Lug bearing 5416 forms the base of cutter head assembly 5332, shown in FIGS. 6A-6C. As subsequently described in further detail, various different cutter heads may alternately be inserted into and secured within the slot shaped opening in the distal end of the lug bearing.

FIGS. 7A-7F show further details of an exemplary rotor housing assembly 5420′. Assembly 5420′ is constructed and operates in a manner similar to assembly 5420 as previously described in reference to FIGS. 6A-6C, but has a different blade configuration. As shown in FIG. 7A, rotor housing assembly 5420′ includes a pair of rotors 5610′ and 5612′, each rotatably mounted in rotor housing 5426 by an axle 5618. In this embodiment, rotors 5610′ and 5612′ are configured to rotate in opposite directions to draw tissue into a center, overlapping region where the tissue is shredded.

Referring to FIGS. 7B and 7C, the components of rotor housing assembly 5420′ are shown. Assembly 5420′ includes housing 5426, a pair of axles 5418, and gears 5410, 5620 and 5622, as previously described. Rotor 5610′ includes two blades 5710 interspersed with three spacer rings 5714 on first axle 5418. Rotor 5612′ includes three blades 5712 interspersed with two spacer rings 5716 on second axle 5418.

It should be noted that while rotor housing assembly 5420′ is shown in an exploded format for clarity in FIGS. 7B and 7C, suggesting that the components are fabricated separately and then assembled using traditional assembly processes, this may or may not be the case, depending on the embodiment. In some embodiments, rotor assembly 5420′ is assembled this way. In other embodiments, assembly 5420′ may be built in layers, such as by using a MEMS fabrication processes. For example, after portions of housing 5426 and gears 5410, 5620 and 5622 are built up in layers, bottom blade 5712, bottom spacer 5714, and housing fin 5624 are formed together in one or more layers. Following this layer, bottom blade 5710, bottom spacer 5716, and bottom housing fin 5626 may be formed together in one or more layers. The process may be repeated until the entire rotors 5610′ and 5612′ and surrounding components are formed. A thin sacrificial layer may be formed between adjacent layers of components to separate the components from one layer from components of adjacent layers. Sacrificial material may also be formed in portions of each non-sacrificial layer to separate components on that layer, create desired voids in the finished assembly, and to provide a substrate for forming components in subsequent layers above. With such a fabrication technique, rotor 5610′ may be formed as a single unitary structure interleaved with portions of rotor housing 5426, rather than separate components (i.e. axle 5418, spacers 5714, blades 5710, and gear 5620.) Similarly, rotor 5612′ may be formed as a single unitary structure interleaved with portions of rotor housing 5426, rather than separate components (i.e. axle 5418, blades 5712, spacers 5716, and gear 5622.) In some embodiments, combinations of fabrication and assembly techniques may be used to create the rotor housing and/or cutter head assemblies.

Referring to the top view shown in FIG. 7D, it can be seen that in this embodiment the axle 5418 of rotor 5612′ is more distally located than axle 5418 of rotor 5610′. It can also be seen that while a top plate portion of rotor housing 5426 covers most of rotor blades 5710 and 5712, the blades protrude less from a middle and bottom plate portion of housing 5426. Further details of protruding blades and rotor characteristics are subsequently discussed in reference to FIG. 7F.

A front or distal end view is shown in FIG. 7G. As depicted in FIG. 7G, very small gaps or interference fits 5717 between overlapping blades 5710 and 5712 are desirable in some embodiments. Similarly, very small gaps or interference fits 5719 between blades 5712 and adjacent portions of rotor housing 5426 are desirable in some embodiments, as will be subsequently described in more detail.

Referring to the cross-sectional plan view of FIG. 7F, the bottom two blades 5712 of rotor 5612′ and the bottom blade 5710 of rotor 5610′ are shown. As shown, blades 5710 have a larger outer diameter than that of blades 5712. But because axle 5418 of rotor 5612′ is located more distally than axle 5418 of rotor 5610′, blades 5712 protrude more distally from the bottom of rotor housing 5426 than do blades 5710 of rotor 5610′. It can also be seen that teeth 5718 and associated troughs 5720 of blades 5712 are configured to be rotationally out of phase with those of other blades 5712 of rotor 5612′. As will subsequently be discussed in more detail, this arrangement can tune rotors 5612 to selective cut certain types of tissue and avoid cutting other types of tissue.

Various rotor gaps can be seen in FIG. 7F. For example, gap 5722 is shown between the tips of blade teeth 5718 of rotor 5612′ and spacer ring 5714/axle 5418 of opposing rotor 5610′. Gap 5724 is also shown, between the tips of blade teeth 5718 of rotor 5612′ and the adjacent portion of housing 5426. Gap 5726 is also shown, between spacer ring 5714/axle 5418 of rotor 5610′ and the adjacent portion of housing 5426. In some embodiments, it is desirable to keep gaps 5722, 5724 and 5726 very small, to ensure that tissue portions/particles that pass through rotors 5610′ and 5612′ are first cut to a very small size, and to avoid jamming or clogging rotors 5610′ and 5612′. In some embodiments, these gaps are fabricated as small interferences between the adjacent parts so that when the rotors are first rotated, the adjacent parts hit each other and wear down or burnish each other. In this manner, after a break in period, smaller interference or zero clearance fits are created between the adjacent moving parts. Gap distances that applicants believe are advantageous include less than about 20 microns, less than about 10 microns, less than about 5 microns, less than about 1 micron, substantially zero, an initial interference fit of at least 2 microns, and an initial interference fit of about 5 microns.

In operation, the cutter elements of rotor housing assembly shown in FIGS. 7A-7F serve to grab tissue from a target source, draw the tissue towards a central region between the blades, cut the tissue from the source, and morcellate the tissue in small pieces for transport away from the body. In other embodiments, separate cutter elements may be used for these various functions. For example, one blade or blades may be used to cut tissue from the source, while another blade or set of blades may be used to morcellate the cut tissue.

Components of cutter head assembly 5332, including rotor housing assemblies 5420 and 5420′, may be fabricated using processes such as laser cutting/machining, photo chemical machining (PCM), Swiss screw, electro-discharge machining (EDM), electroforming and/or other processes for fabricating small parts. Wafer manufacturing processes may be used to produce high precision micro parts, such as EFAB, X-ray LIGA (Lithography, Electroplating, and Molding), and/or UV LIGA. An electrochemical fabrication technique for forming three-dimensional structures from a plurality of adhered layers is being commercially pursued by applicant Microfabrica® Inc. (formerly MEMGen Corporation) of Van Nuys, Calif. under the name EFAB®. Such a technique may be advantageously used to fabricate components described herein, particularly rotors and associated components.

In some embodiments, the shredder's ability to selectively remove tissue is attributed to the protrusion of the rotating cutters from the housing and the design of a tooth pitch (space between the tips of adjacent teeth) of each rotor. In some embodiments, the protrusion sets the depth of the inward cut for the tips of the rotor. This inward depth controls the thickness of tissue being removed. The tooth pitch or number of teeth circumferentially about the rotor diameter provides an opening for individual tissue fibers and/or fiber bundles to be hooked, tensioned and drawn between the cutters.

From the point of view of the selected tissue, the tooth pitch and protrusion may be designed to grasp the smallest fibers or fiber bundles that are to be removed. From the point of view of the non-selected tissue, the tooth pitch may be many times smaller than the fiber or fiber bundle, and the protrusion may also be equally smaller than the fiber/bundle diameter.

As previously described, FIG. 7D shows the exemplary protrusion of blades 5710 and 5712 as viewed from the top of a rotor housing assembly 5420′. In some embodiments, the protrusion is more exposed on the top side than the bottom. In other embodiments, the cutter device has the same protrusion for both sides. Biasing the protrusion more on one side than the other can provide advantages such as cutting/shredding directionality and/or additional safety. Blade protrusion distances that applicants believe are advantageous include less than about 100 microns, less than about 10 microns, substantially flush with the housing, recessed a minimum of about 5 microns, and recessed a minimum of about 10 microns.

Tooth pitch is the distance from one tooth tip to the next tooth tip along an imaginary circle circumscribing the outer circumference of the blade. The trough diameter or depth generally is the distance between the tooth tip and the low point between the tooth tips. In many embodiments, the trough is a critical geometry component that enables tissue selectivity. Additionally, the trough opening (i.e. the distance from tooth tip to the tooth back of an adjoining tooth) can determine the size of the “window” for capturing a fiber or fiber bundle diameter.

In some embodiments, the target tissue being cut is hydrated and generally has a nominal fiber diameter of about 6 to about 9 microns. In some embodiments, the target tissue being cut is dry and generally has a nominal fiber diameter of about 5 to about 6 microns. In some embodiments, the tissue fibers are connected together in bundles having a nominal diameter of about 250 microns.

Typical dimensions in some embodiments include:

Housing diameter: 6 mm or less

Blade diameter range: 0.75 mm to 4 mm

Tip to Tip range: 0.2 mm to 1 mm

Trough diameter range: 2 microns to 0.5 mm

Blade protrusion range: 2 microns to 2 mm

The tip to tip distance is typically at least two times the trough diameter for hook type teeth.

The tissue cutting devices disclosed herein may be configured for use in a variety of procedures. An example of a cardiac application is using the inventive devices to selectively remove endocardium, with the cutting device configured to leave the underlying myocardium uncut. An example of a tissue removing application involving the esophagus includes selectively removing mucosa, leaving the submucosa. Such a therapy would be useful for treating Barrett's disease. Examples in the spinal area include selectively removing flavum, with the cutting device configured to stop removing tissue when dura is reached, leaving the dura intact. Selective removal of flavum but not nerve root is another embodiment. A cutting device constructed according to aspects of the invention can also be configured to remove flavum without cutting bone. In this embodiment, the rotor velocity could be changed and/or the cutting elements could be changed after the flavum is removed such that some bone tissue could then be removed. Examples in the neurovascular area include selectively removing cancerous tissue while not cutting adjacent blood vessel tissue or nerve tissue. In the rheumatology field, tears in labral target tissue may be selectively removed while preserving adjacent non-target tissue, such as in the hips, shoulders, knees, ankles, and small joints. In some embodiments, small teeth on the rotors can interact with micron scale fibers of cartilage, removing tissue in a precise way, much like precision machining of materials that are harder than tissue. Other target tissues that may be selectively removed by the inventive devices and methods described herein include cartilage, which tends to be of a medium density, periosteum, stones, calcium deposits, calcified tissue, cancellous bone, cortical bone, plaque, thrombi, blood clots, and emboli.

It can be appreciated by those skilled in the art of tissue removal that soft tissue is much more difficult to remove in a small quantities and/or in a precise way than harder tissue such as bone that may be grinded or sculpted, since soft tissue tends to move or compress when being cut, rather than cut cleanly. Cutting tissue rather than removing it with a laser or other high energy device has the advantage of not overheating the tissue. This allows the tissue to be collected and its pathology tested, as previously described.

In some embodiments of the invention, the selective tissue cutting tool may be moved laterally along a tissue plane, removing thin swaths of tissue with each pass until the desired amount or type of tissue is removed. In some embodiments, the tool may be plunged into the target tissue in a distal direction, until a desired depth or type of tissue is reached. In any of these embodiments, the tool may cut a swath or bore that is as large as or larger than the width of the tool head. In some embodiments, the cutting elements are distally facing, laterally facing, or both.

According to further aspects of the present disclosure, the rotational axis or axes of a single or dual rotor cutter can be located and angled in three-dimensional space in a variety of configurations relative to a longitudinal axis of the debrider device to allow access to target tissue sites not accessible by conventional debriders. These unique configurations enable medical procedures that otherwise could not be performed, or permit the procedures to be performed more easily.

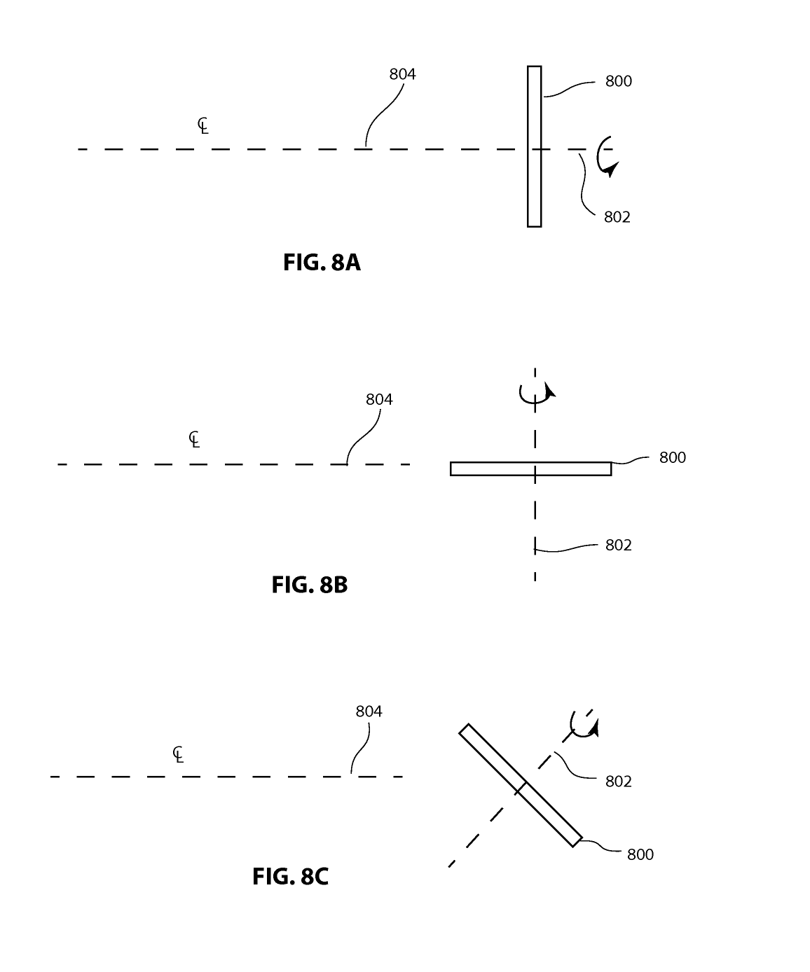

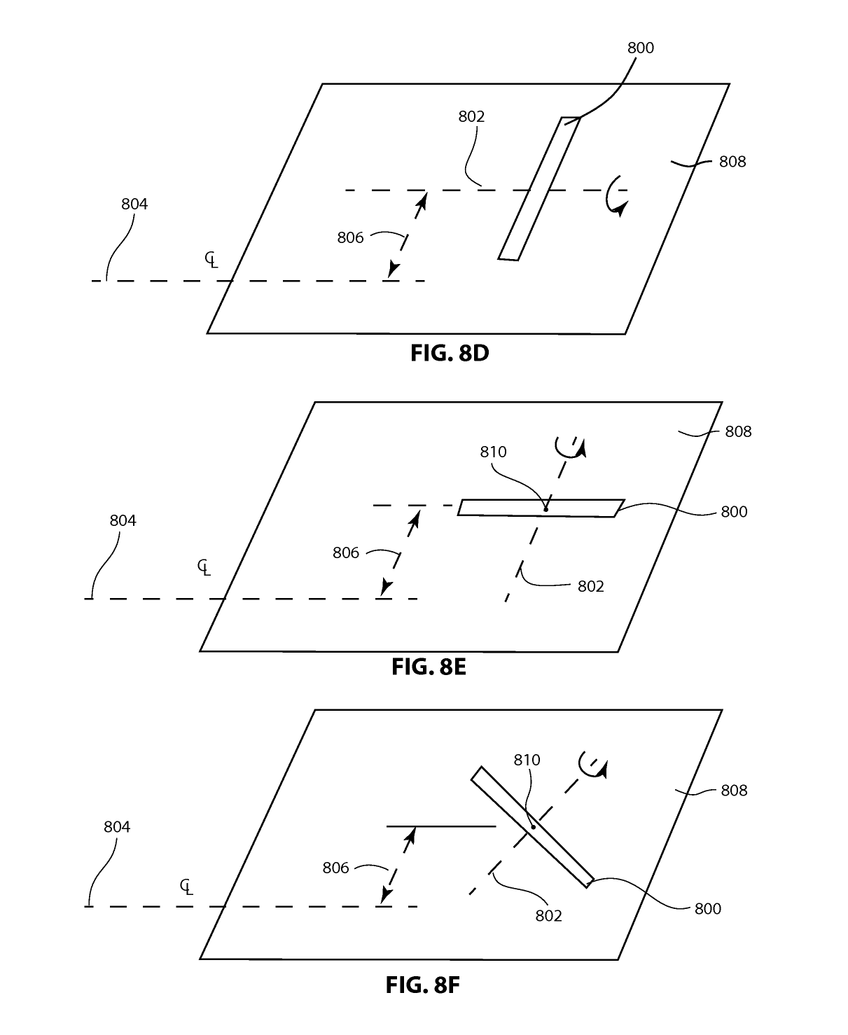

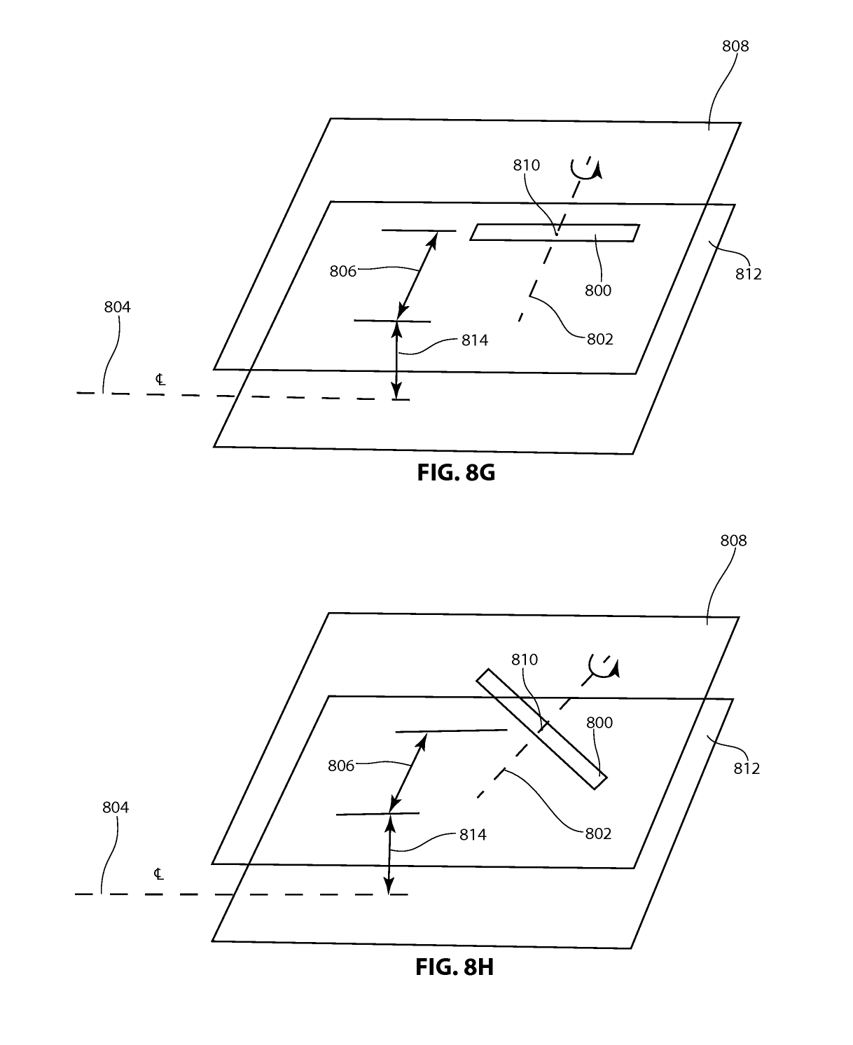

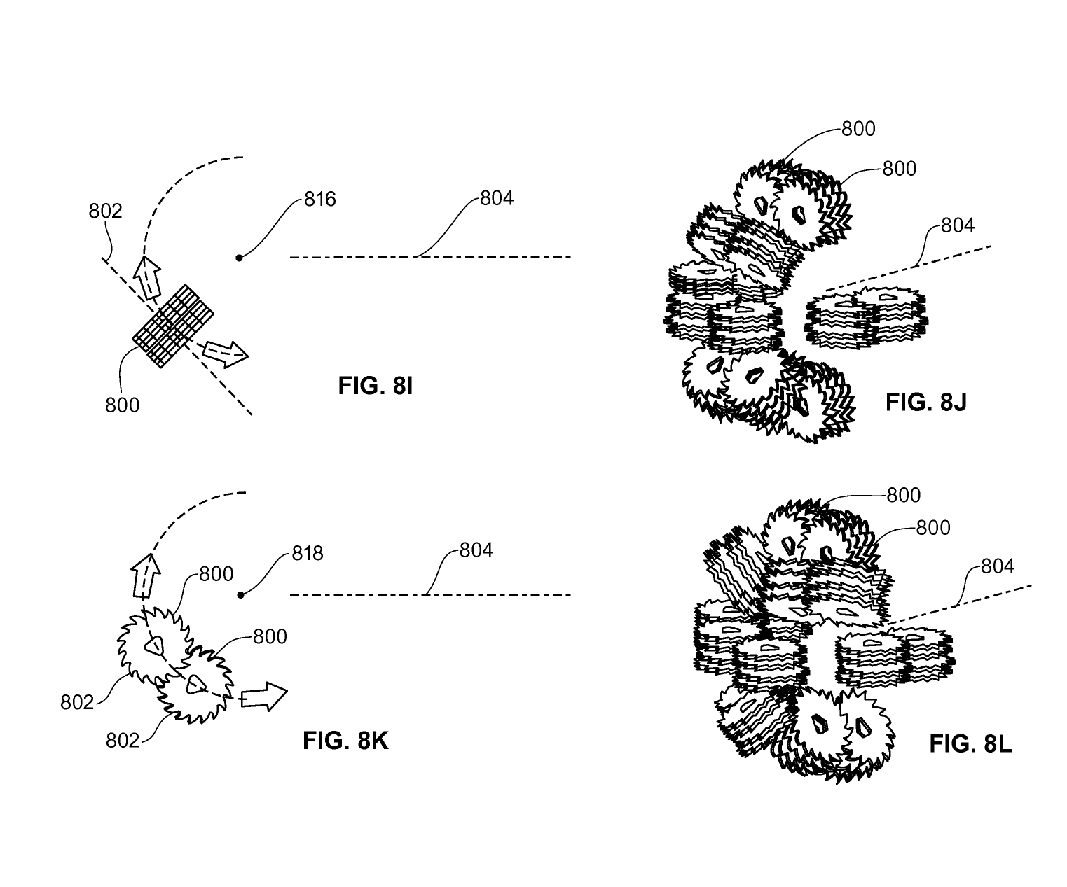

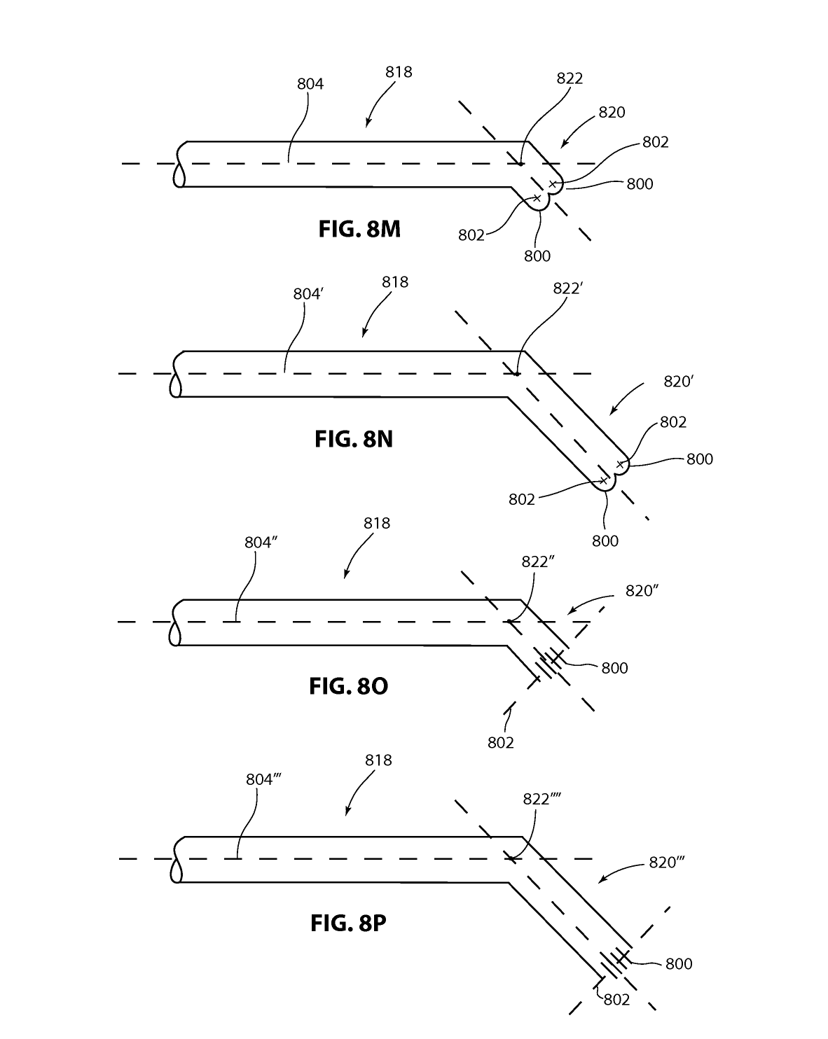

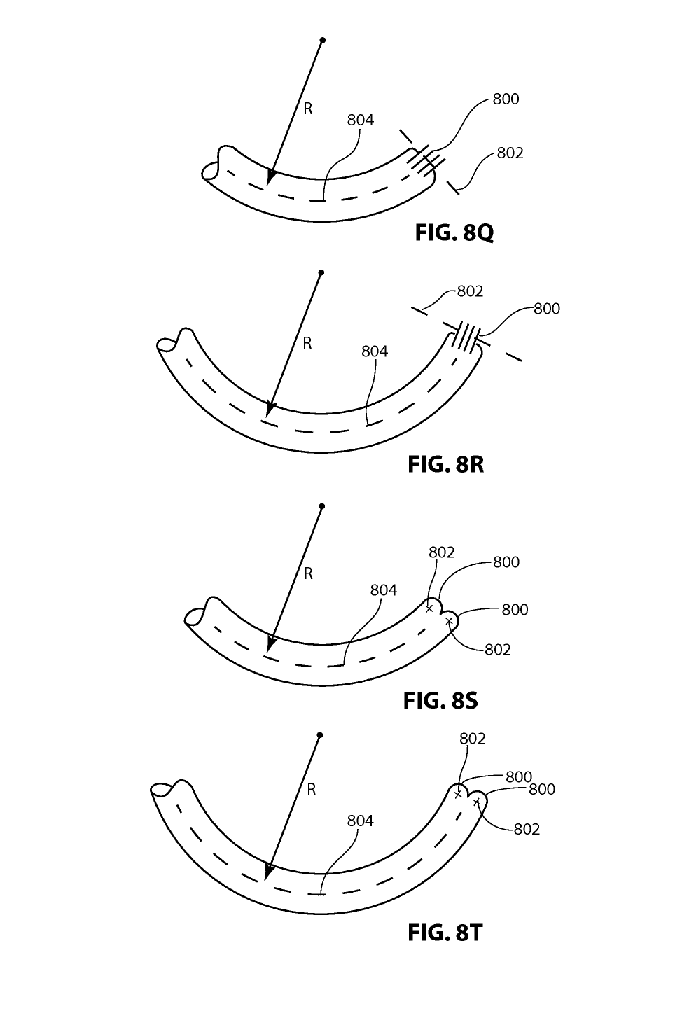

Referring to FIGS. 8A-8U, various rotor orientations enabled by the present disclosure are schematically shown. For clarity of illustration and explanation, the rotors depicted in these figures are shown with only a single blade without any tooth detail. Functional surgical instruments may be fabricated with these simplified constructs. However, the concepts being discussed relative to these embodiments may be equally applied to the other embodiments disclosed herein (e.g., rotors having many blades and/or single or multi-toothed blades.) Additionally, various portions of the rotors depicted in the figures may be shown as separate components for clarity. In some embodiments, these portions may be fabricated as separate components, while in other embodiments they may be integrally formed into unitary rotors.

Referring first to FIG. 8A, an edge view of a single rotatable member 800 depicted with a single blade (such as blade 5710 of FIG. 7B) is shown having a rotational axis 802. A central longitudinal axis 804 of an elongate member is also depicted. Axis 804 is typically the central axis of an outer shaft, such as outer support tube 5328 shown in FIG. 5B. In some embodiments, axis 804 is the central axis of a distal most portion of a debrider shaft, which itself may be angled with respect to a proximal portion of the debrider shaft.

As shown in FIG. 8A, rotational axis 802 of rotatable member 800 may be configured to be coincident with longitudinal axis 804. This configuration of is often referred to as an end cutter. A detailed disclosure of exemplary end cutter embodiments is provided in copending U.S. Patent Publication No. 2012/0191121 published Jul. 26, 2012 and entitled Concentric Cutting Devices For Use In Minimally Invasive Medical Procedures. Drive train systems for such embodiments can be fairly simple, using a drive tube connected directly to the rotatable member(s).

As shown in FIG. 8B, rotational axis 802 of rotatable member 800 may be configured to intersect with longitudinal axis 804 and be perpendicular therewith. Drive train systems for such embodiments may utilize gear systems similar to those described in conjunction with previously described embodiments, and will be subsequently described in more detail.

As shown in FIG. 8C, rotational axis 802 of rotatable member 800 may be configured to intersect with longitudinal axis 804 and form an angle therewith of between 0 and 90 degrees.

As shown in FIG. 8D, rotational axis 802 of rotatable member 800 may be configured to be parallel to longitudinal axis 804 and offset from it by a predetermined distance 806. In this embodiment, rotational axis 802 and longitudinal axis 804 lie in a common plane 808.

As shown in FIG. 8E, rotational axis 802 of rotatable member 800 may be configured to be perpendicular to longitudinal axis 804. In this embodiment, rotational axis 802 and longitudinal axis 804 lie in a common plane 808. A center point 810 of rotatable member 800 is offset from longitudinal axis by a predetermined distance 806 in plane 808. In the single blade embodiments depicted, center point 810 lies on rotational axis 802 halfway between the top and bottom surfaces of the blade if both surfaces are used for shearing tissue. If only one surface of rotatable member 800 is used for shearing tissue, the center point 810 lies on that surface where rotational axis 802 passes through it. For multi-blade rotatable members, the center point is on the rotational axis 802 halfway between the outermost tissue shearing surfaces of rotatable member 800.

As shown in FIG. 8F, rotational axis 802 of rotatable member 800 may be configured to form an angle with longitudinal axis 804 of between 0 and 90 degrees. In these embodiments, rotational axis 802 and longitudinal axis 804 lie in a common plane 808. A center point 810 of rotatable member 800 is offset from longitudinal axis by a predetermined distance 806 in plane 808.

As shown in FIG. 8G, rotational axis 802 of rotatable member 800 may be configured to be perpendicular to longitudinal axis 804. In this embodiment, rotational axis 802 lies in plane 808 and longitudinal axis 804 lies in a different plane 812 which is offset from plane 808 by a predetermined distance 814. A center point 810 of rotatable member 800 is offset by a predetermined distance 806 in plane 808 from a projection of longitudinal axis 804 onto plane 808. The tissue cutter head embodiments of FIGS. 1-7 provide examples of this configuration. Each of the two blade stacks or rotatable members in these embodiments has a rotational axis that is offset from and perpendicular to the longitudinal axis of the elongate member and lies in a different plane. It should be noted that in some embodiments, the offset distance 806 in plane 808 can be zero. In other words, the center point 810 of one or more rotatable members 800 may be in line with the longitudinal centerline 804 of the elongate member of the debrider, but offset laterally from it by a distance 814. In some embodiments having multiple rotatable members 800, the offset distance 814 from the longitudinal centerline 804 is the same for each rotational member 800. In other embodiments, the multiple rotatable members 800 can have different offset distances 814 from the longitudinal centerline 804.

As shown in FIG. 8H, rotational axis 802 of rotatable member 800 may be configured to form an angle with longitudinal axis 804 of between 0 and 90 degrees. In these embodiments, rotational axis 802 lies in plane 808 and longitudinal axis 804 lies in a different plane 812 which is offset from plane 808 by a predetermined distance 814. A center point 810 of rotatable member 800 is offset by a predetermined distance 806 in plane 808 from a projection of longitudinal axis 804 onto plane 808. Again, the offset distance 806 in plane 808 may be zero in some embodiments. In some embodiments having multiple rotatable members 800, the offset distance 814 from the longitudinal centerline 804 is the same for each rotational member 800. In other embodiments, the multiple rotatable members 800 can have different offset distances 814 from the longitudinal centerline 804.

Referring to FIGS. 8I-8L, tissue cutting heads that are configured to articulate are shown. While the use of various arrangements is shown with dual rotatable members, single rotatable member cutting heads may be used instead. In each of these embodiments shown, the rotational axis 802 of rotatable member 800 is at least initially perpendicular to the longitudinal axis 804 of the elongate member and is configured to articulate with respect thereto. In operation, the rotatable members 800 are articulated by loosening a locking mechanism (not shown), moving the rotatable members 800 to a desired orientation, tightening the locking mechanism, and then using the rotatable members 800 to cut tissue. Drivetrains employing micro gears, as previously described, may be employed to allow the cutting heads to be driven in any of the orientation shown.

As shown in FIG. 8I, which is a side view, rotational axis 802 of rotatable members 800 may be pivoted about an articulation axis 816 that is perpendicular to rotational axis 802. This movement produces a series of positions, either infinite or discrete, that extend from above the distal end of the elongate member to below the distal end of the elongate member, as exemplified by the five positions in a vertical plane shown in FIG. 8J.

As shown in FIG. 8K, which is a top view, rotational axis 802 of rotatable members 800 may be pivoted about an articulation axis 818 that is parallel to rotational axis 802. This movement produces a series of positions, either infinite or discrete, that extend from one side of the distal end of the elongate member to the other side of the distal end of the elongate member, as exemplified by the three positions in a horizontal plane shown in FIG. 8J. As shown in FIG. 8L, rotational axis 802 of rotatable members 800 may be pivoted about both articulation axis 816 and articulation axis 818. This movement produces a series of positions, either infinite or discrete, that define a hemisphere at the distal end of the elongate member, as exemplified by all nine positions shown in FIG. 8L. The mechanism for articulation may include a hinge type joint having a pivot running through the housing that holds the blades to the cutter head and intersecting the outer tube. The motion for articulation may be accomplished by tensioning pull wires.

Referring to FIGS. 8M-8P, distal portions of debrider devices are shown having elongate members 818 comprising a distal portion 820 that is oriented at an angle with respect to a more proximal portion of the elongate member such that the central longitudinal axis 804 has an inflection point 822 between the distal portion 820 and more proximal portion.

As shown in FIG. 8M, distal portion 820 of the central longitudinal axis 804 and a more proximal portion of the central longitudinal axis 804 lie in a common plane that is generally perpendicular to the rotational axes 802, 802 of the rotatable members 800. Similarly, as shown in FIG. 8N, distal portion 820′ of the central longitudinal axis 804′ and a more proximal portion of the central longitudinal axis 804′ lie in a common plane that is generally perpendicular to the rotational axes 802, 802 of the rotatable members 800. In some embodiments, the distal portion 820 extends laterally from the elongate member 818 by a distance that is less than a diameter of the elongate member 818, as depicted in FIG. 8M. In other embodiments, the distal portion 820′extends laterally from the elongate member 818 by a distance that is greater than a diameter of the elongate member 818, as depicted in FIG. 8N. In various embodiments, the angle formed between the distal portion 820 or 820′ of the central longitudinal axis 804 and a more proximal portion of the central longitudinal axis 804 is between 0° and about 90°. In some embodiments, the angle formed is greater than 90°.

As shown in FIG. 80, distal portion 820″ of the central longitudinal axis 804″ and a more proximal portion of the central longitudinal axis 804″ lie in a common plane that is coincident with or generally parallel to the rotational axes 802, 802 of the rotatable members 800. Similarly, as shown in FIG. 8P, distal portion 820′″ of the central longitudinal axis 804′″ and a more proximal portion of the central longitudinal axis 804′″ lie in a common plane that is coincident with or generally parallel to the rotational axes 802, 802 of the rotatable members 800. In some embodiments, the distal portion 820″ extends laterally from the elongate member 818 by a distance that is less than a diameter of the elongate member 818, as depicted in FIG. 8O. In other embodiments, the distal portion 820′″ extends laterally from the elongate member 818 by a distance that is greater than a diameter of the elongate member 818, as depicted in FIG. 8P. In various embodiments, the angle formed between the distal portion 820″ or 820′″ of the central longitudinal axis 804 and a more proximal portion of the central longitudinal axis 804 is between 0° and about 90°. In some embodiments, the angle formed is greater than 90°.

In some embodiments of the disclosure (not shown), a combination of two or more inflection points 822, 822′, 822″ and/or 822′″ may be utilized to form a multi-segmented elongate member configured to cross specific anatomies to reach target tissue to be removed.

Referring to FIGS. 8Q-8T, distal portions of debrider devices are shown each having elongate members comprising a generally rigid, curved distal portion. These portions are formed on or coupled with a generally straight, more proximal portion (not shown).

As shown in FIGS. 8Q and 8R, the curved, distal portion of the central longitudinal axis 804 may lie in a plane that is coincident with or generally parallel to rotational axis 802 of rotatable member 800.

As shown in FIGS. 8S and 8T, the curved, distal portion of the central longitudinal axis 804 may lie in a plane that is generally perpendicular to rotational axis 802 of rotatable member 800.

In some embodiments, the curved, distal portion sweeps out an arc of less than 90°, as shown in FIGS. 8Q and 8S. In other embodiments, the curved, distal portion sweeps out an arc of more than 90°, as shown in FIGS. 8R and 8T. In some embodiments, the radius of curvature R of the curved distal portion is less than about four times the diameter of the elongate member. In other embodiments, the radius of curvature R of the curved distal portion is more than about four times the diameter of the elongate member.

In some embodiments of the disclosure (not shown), a combination of two or more curved, distal portions, such as those shown in FIGS. 8Q-8T, may be utilized to form a multi-segmented elongate member configured to cross specific anatomies to reach target tissue to be removed. In other embodiments, one or more curved, distal portions, such as those shown in FIGS. 8Q-8T, may be combined with one or more inflection points, such as those shown in the FIGS. 8M-8P.

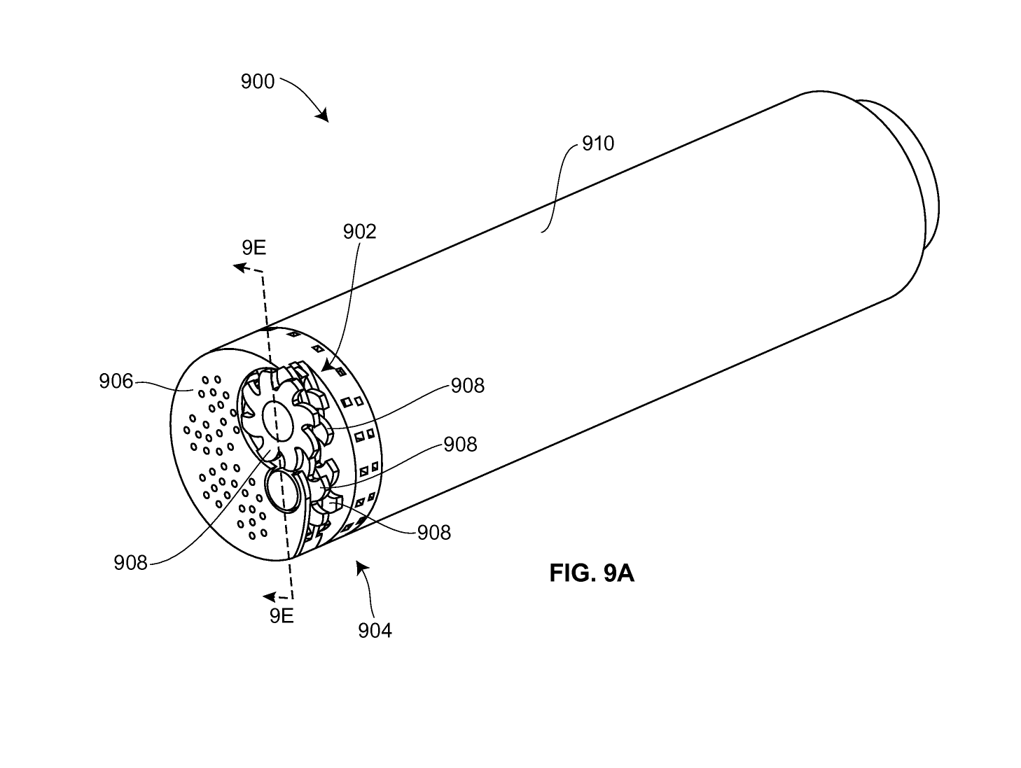

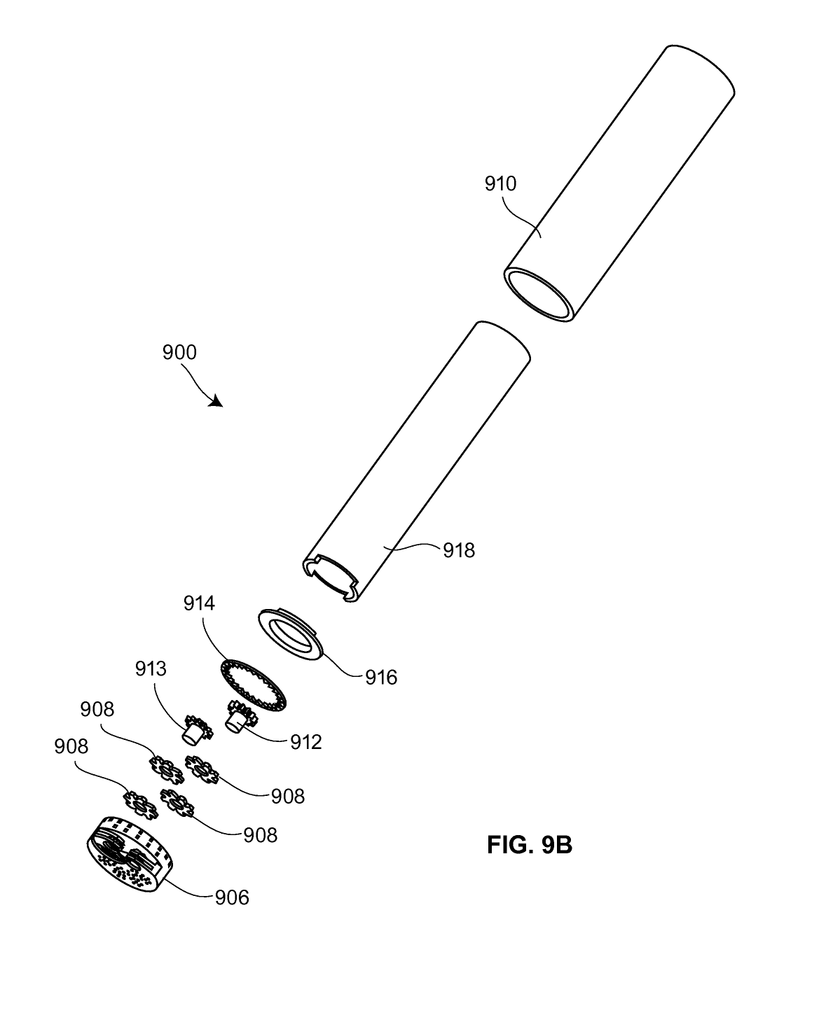

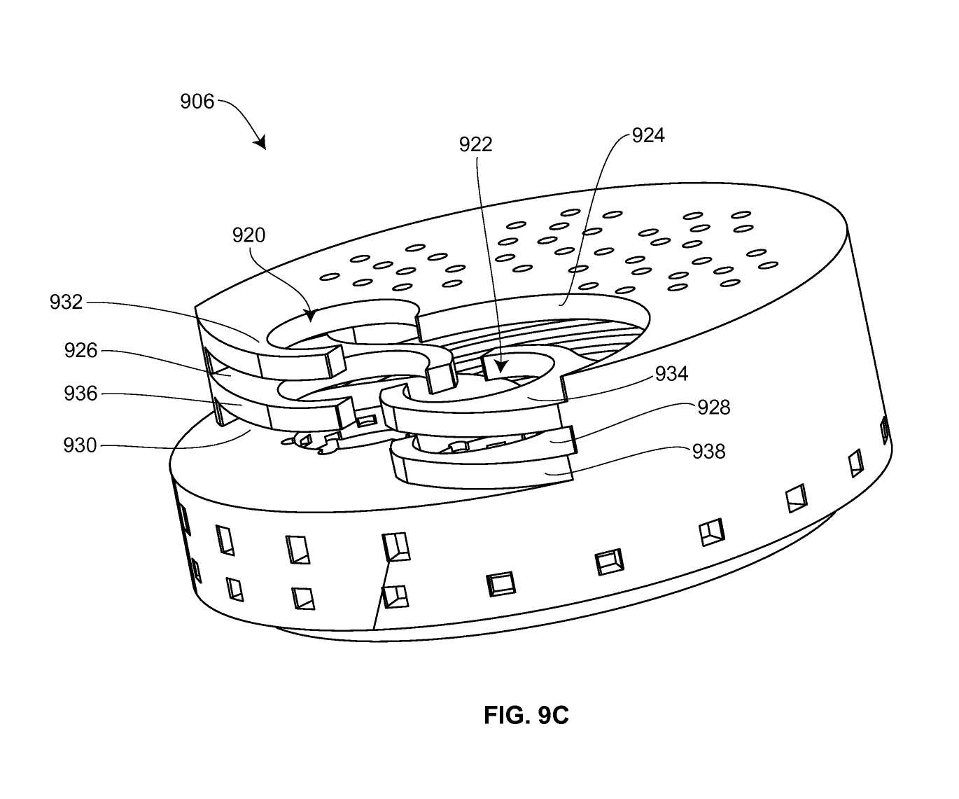

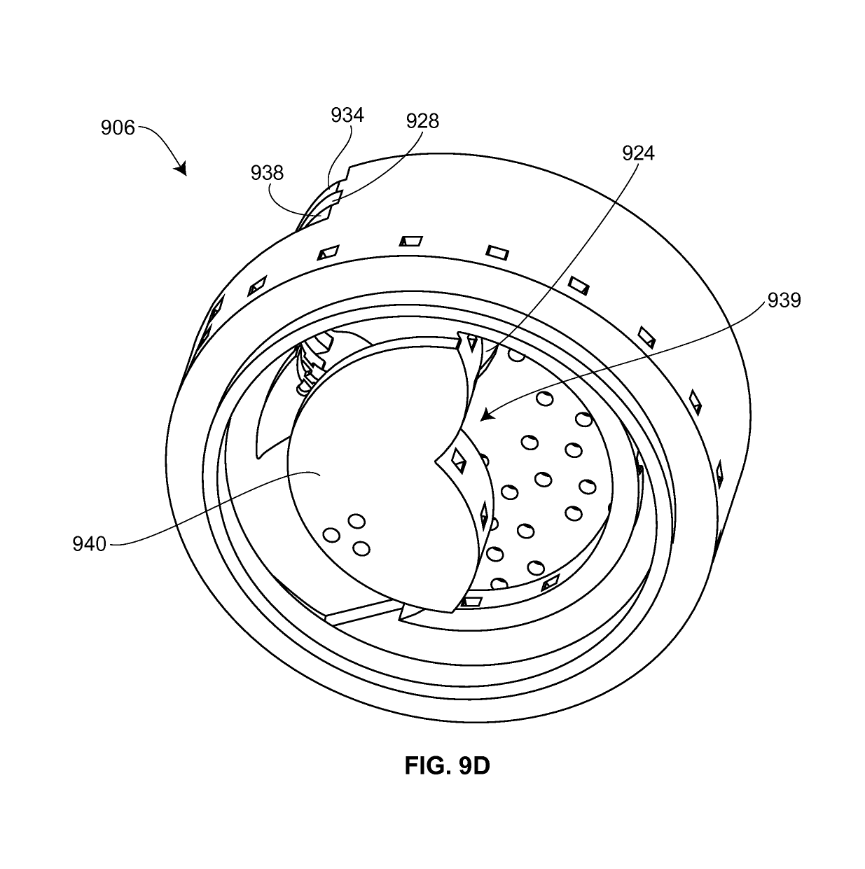

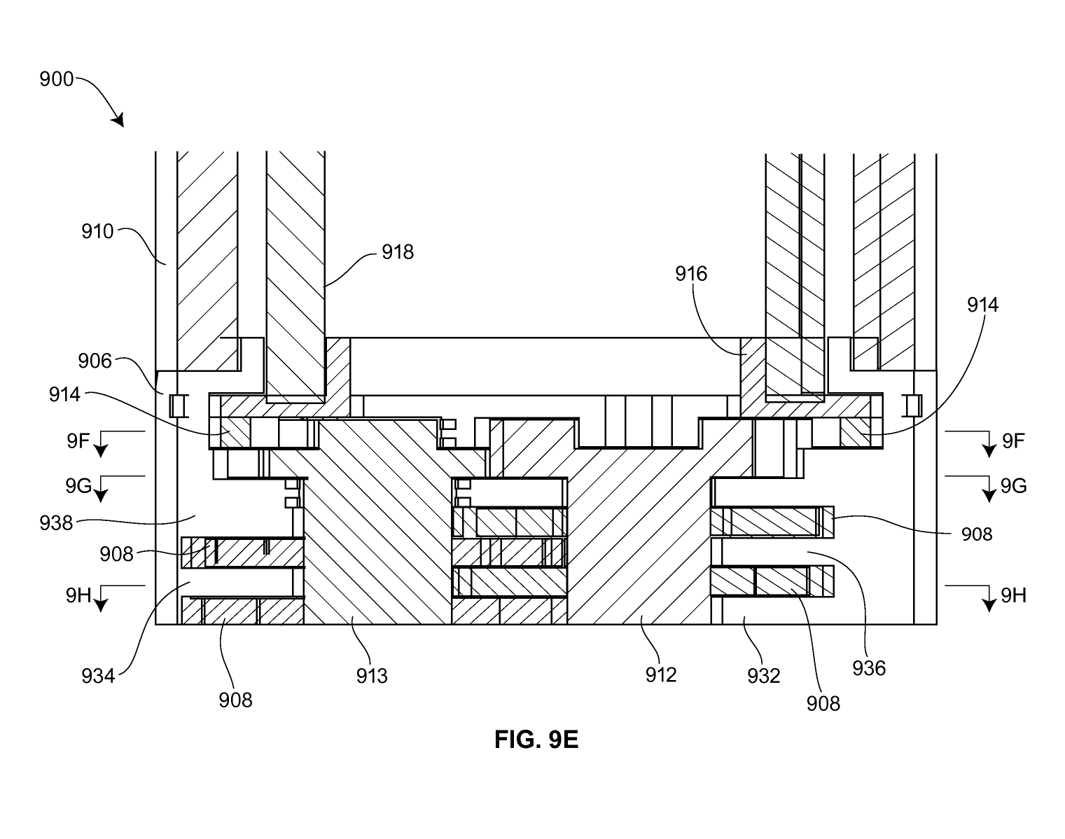

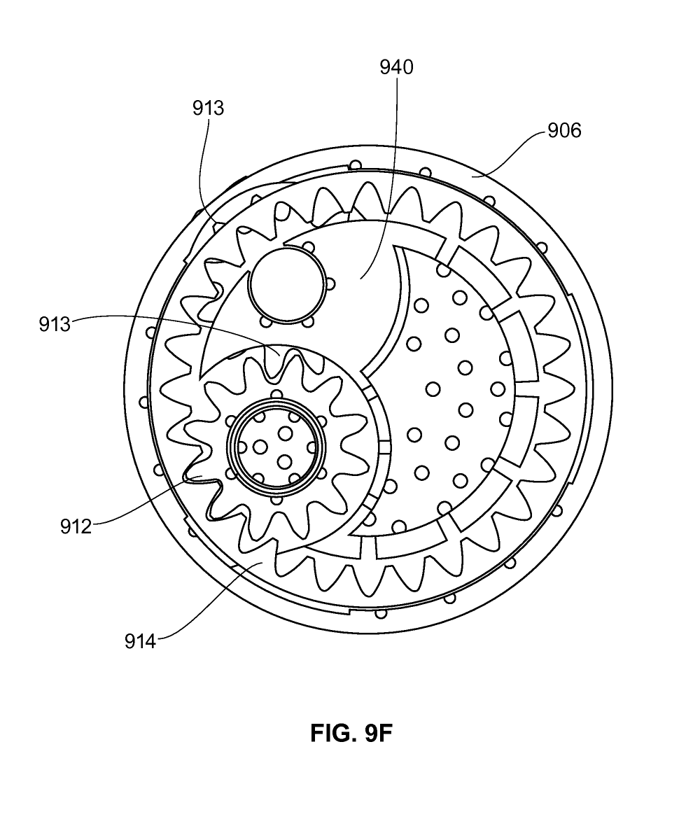

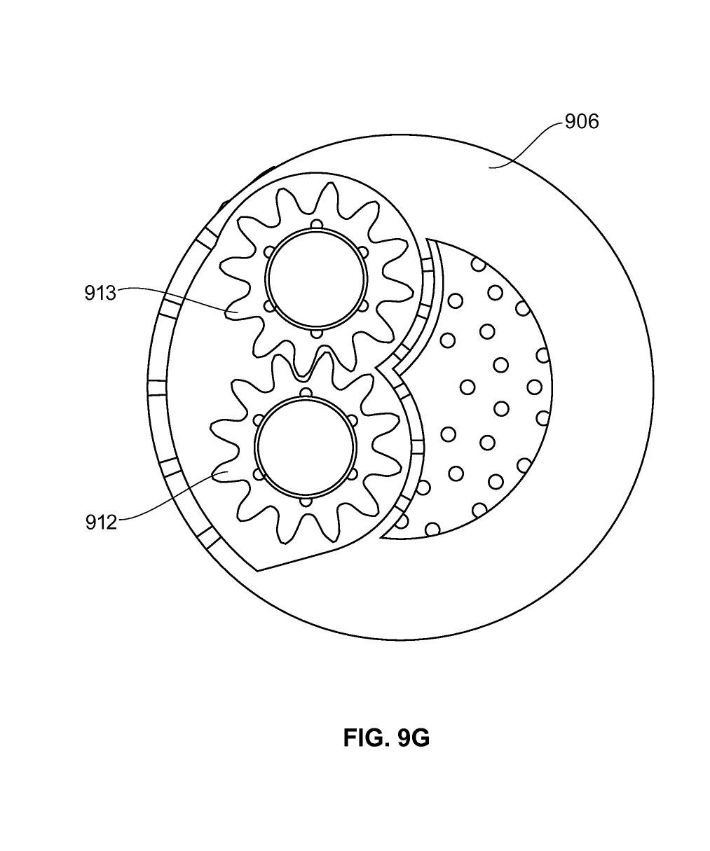

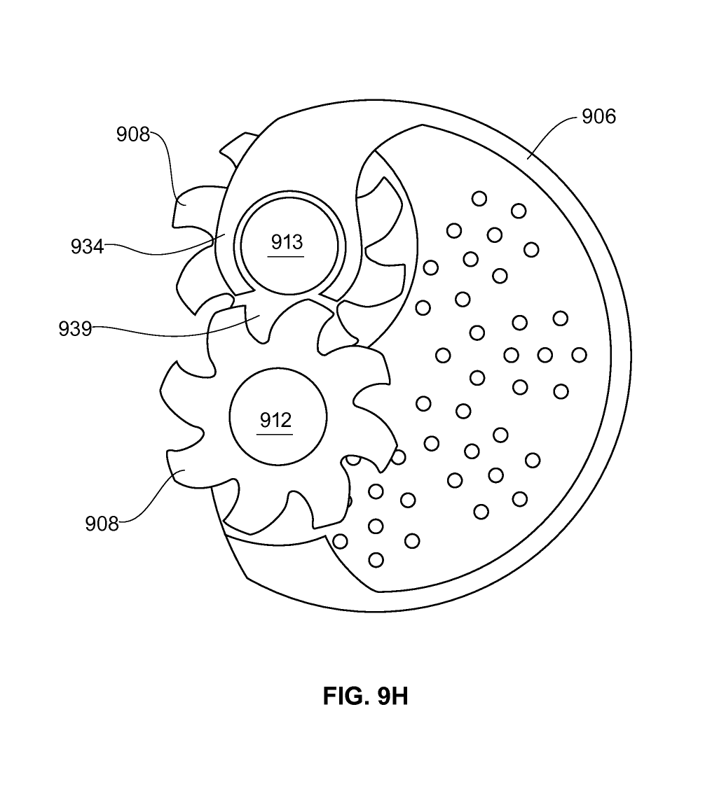

Referring to FIGS. 9A-9H, the distal end of an exemplary embodiment of a side mount tissue shredder 900 is shown. As best seen in FIG. 9A, tissue shredder 900 includes two oppositely rotating members 902 and 904 which are configured to engage tissue from beside the distal end of the shredder 900 and draw the tissue into the distal housing 906 while the tissue is being shredded. In this embodiment, each of the rotatable members 902 and 904 include two cutting blades 908. The rotatable members 902 and 904 are further examples of the type of cutter arrangement depicted in FIG. 8D. More specifically, the rotational axes of rotatable members 902 and 904 are configured to be parallel to a longitudinal axis of shaft 910 and offset from it by a predetermined distance. Each rotational axis lies in a common plane with the longitudinal axis.

As best seen in FIG. 9B, tissue shredder 900 includes a distal housing 906, four blades 908, two twelve-tooth spur gears 912 and 913, a twenty-four tooth internal gear 914, a drive tube ring 916, an inner drive tube 918, and an outer tube 910. In this embodiment, outer tube 910 is rigidly connected to a handle (not shown) at the proximal end of device 900. Inner drive tube 918 rotates concentrically within outer tube 910 to drive the blades 908. The distal end of inner drive tube 918 is castellated to mate with the proximal side of drive tube ring 916. This arrangement allows inner drive tube 918 to rotationally drive the drive tube ring 916 while also allowing some longitudinal movement of drive tube 918 relative to drive tube ring 916. Drive tube ring 916 may be welded, epoxied or otherwise affixed or rotationally coupled with internal gear 914.

Distal housing 906 may be welded, epoxied or otherwise affixed to the distal end of outer tube 910. As shown in FIG. 9C, distal housing 906 includes two laterally spaced bores 920 and 922, each for receiving a shaft of a spur gear 912 and 913. Distal housing 906 also includes four overlapping recesses 924, 926, 928 and 930, each for receiving a blade 908. The four overlapping recesses 924, 926, 928 and 930 are at least partially defined by four C-shaped arms 932, 934, 936 and 938. These C-shaped arms support the shafts of spur gears 912 and 913 and fill the space between the interdigitated blades 908 (shown in FIGS. 9A and 9B). These C-shaped arms also provide tissue shearing surfaces or edges that cooperate with blades 908 to sheer tissue therebetween. A gap 939 is provided through a middle portion of the C-shaped arms to allow sheared tissue pieces to pass through to the center of the distal housing 906. FIG. 9D is a bottom perspective view of distal housing 906 and shows spur gear support structure 940 configured to support the bottom of spur gears 912 and 913.

FIGS. 9E-9H further illustrate the drivetrain used to rotate blades 908. As previously described in reference to FIG. 9B, inner drive tube 918 rotationally drives internal gear 914 through drive tube ring 916. As shown in FIG. 9F, internal gear 914 drives spur gear 912. The teeth of spur gear 913 are thinner than those of spur gear 912 and reside on a more distal plane such that they do not engage with internal gear 914. The teeth of spur gear 912 are thick enough such that they engage with internal gear 914 on a more proximal plane (as shown in FIG. 9F) and with spur gear 913 on a more distal plane (as shown in FIG. 9G). In this way, internal gear 914 drives spur gear 912, which in turn drives spur gear 913. As shown in FIG. 9H, spur gears 912 and 913 each drive two blades 908 which are formed on or affixed to the shafts of the spur gears 912 and 913. FIG. 9H further shows the overlapping, interdigitated arrangement of blades 908, and the gap 939 provided through the center portion of the blades for transporting tissue pieces that have been sheared by the overlapping blades into the open interior portion of the housing.

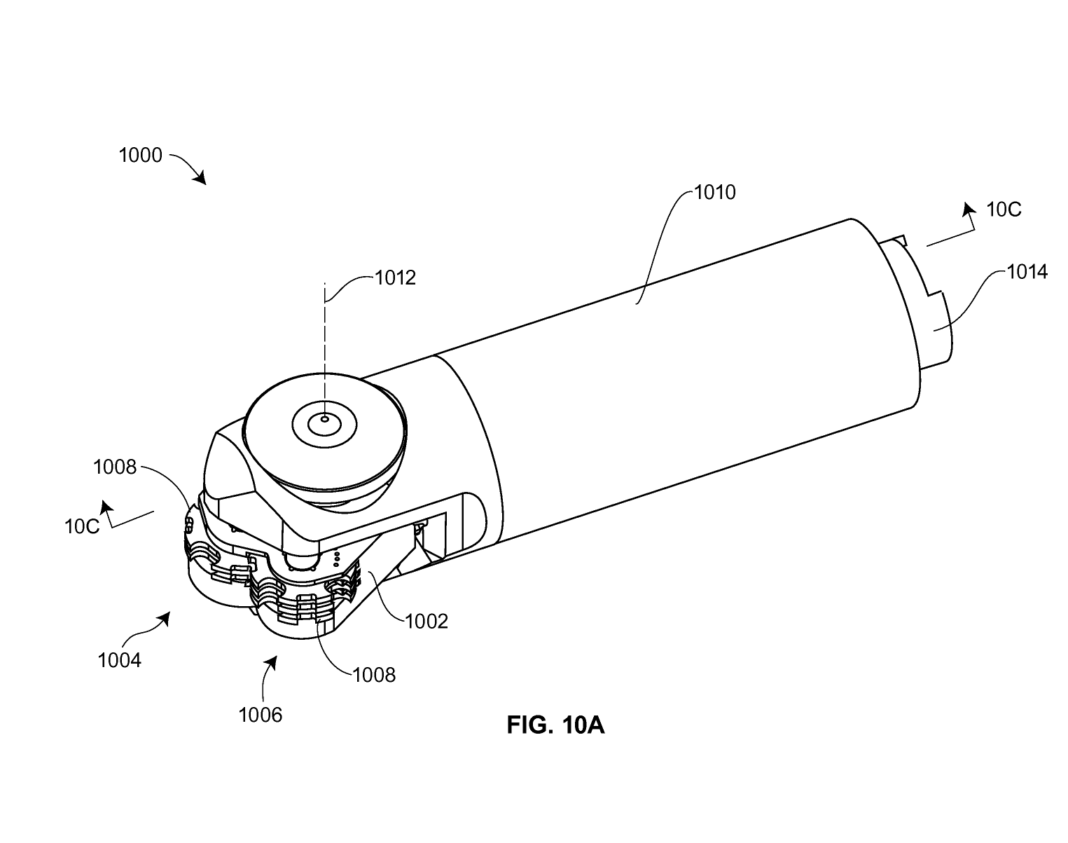

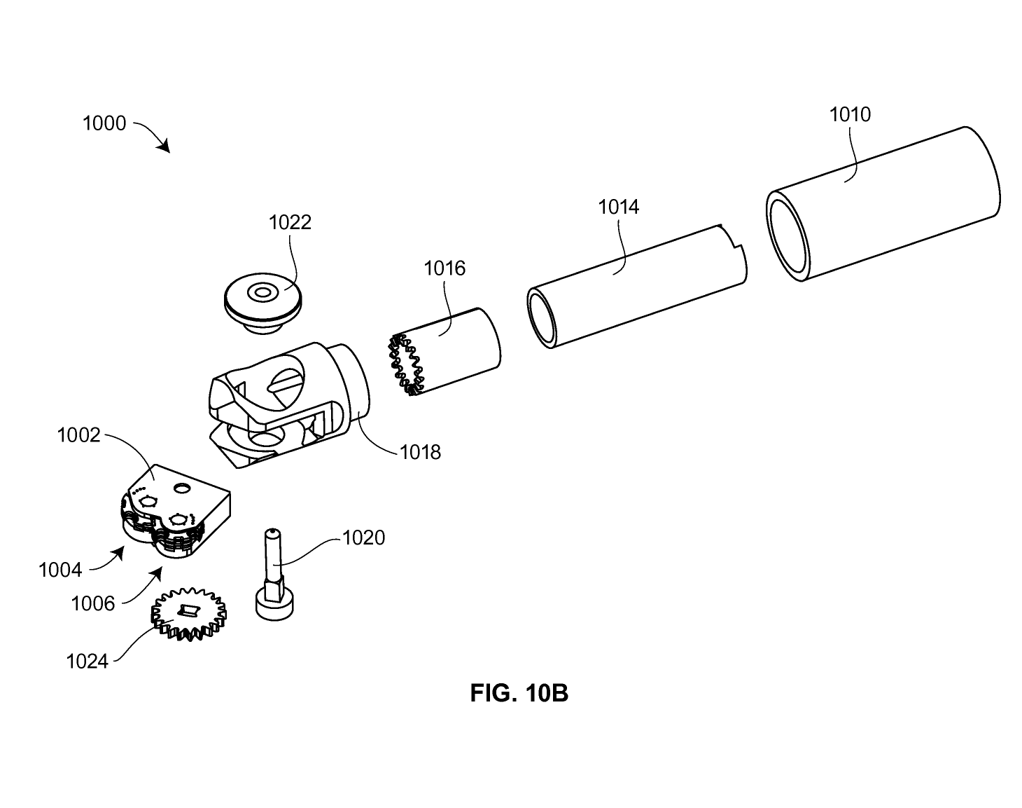

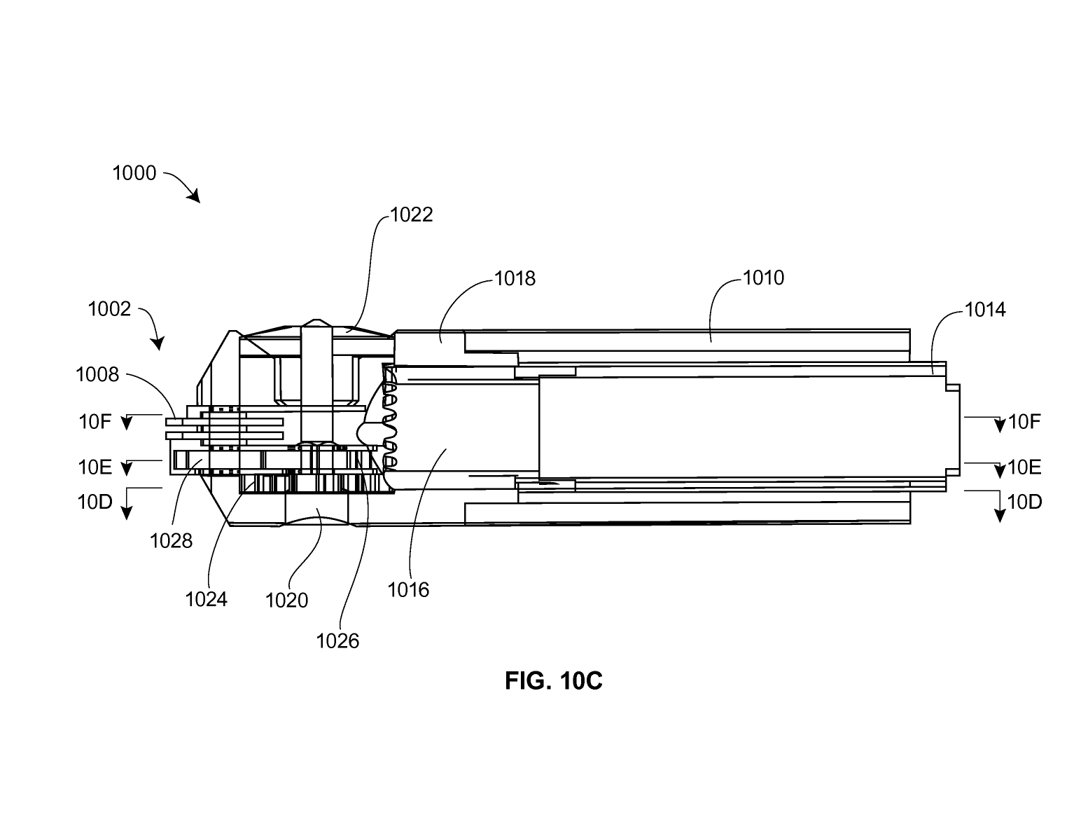

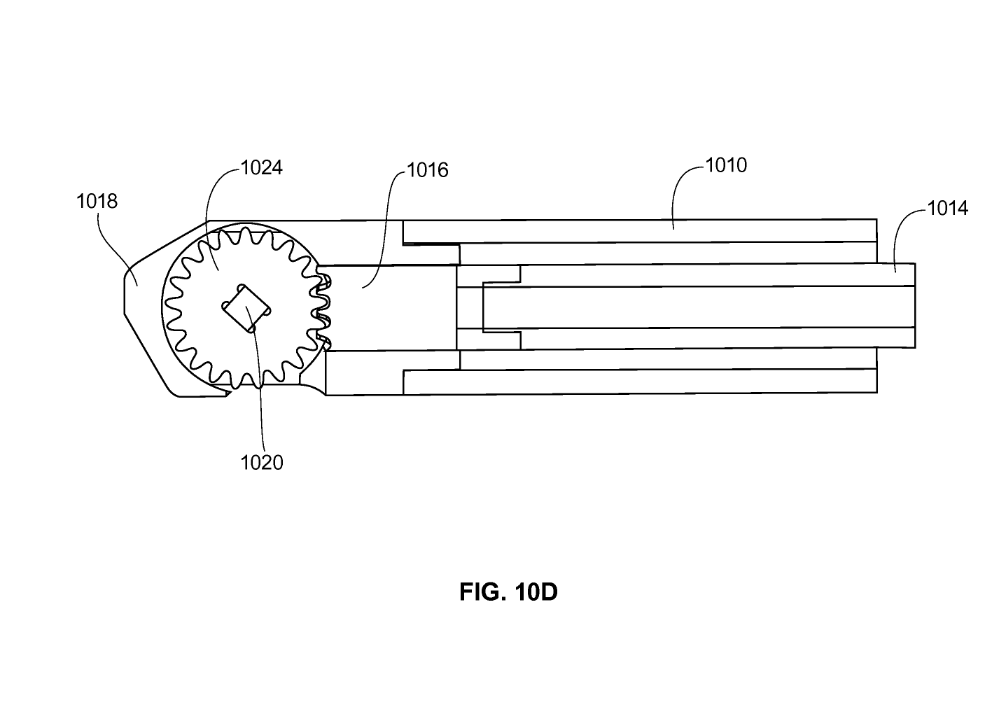

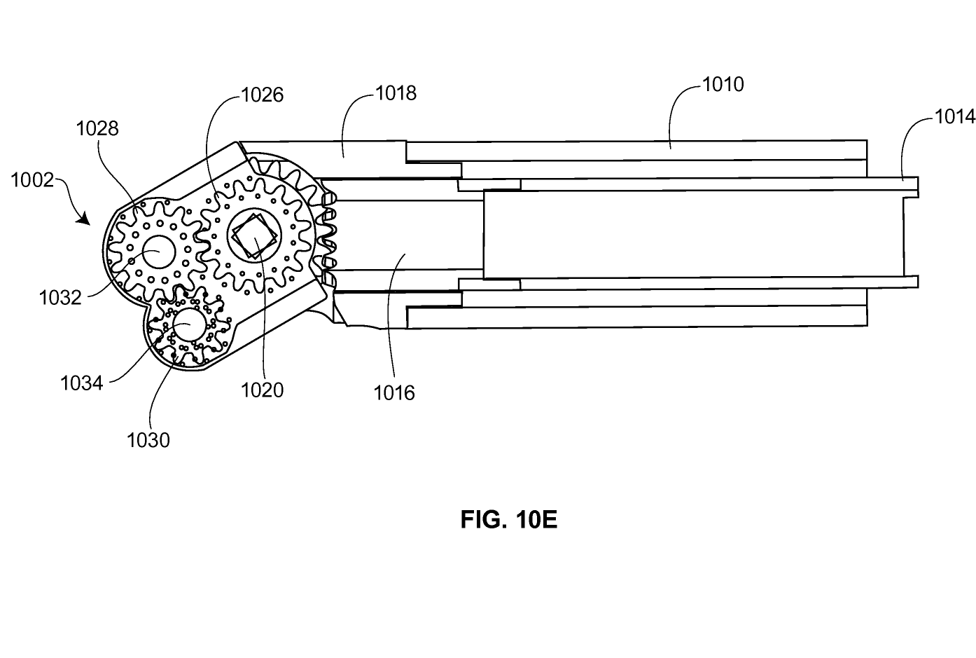

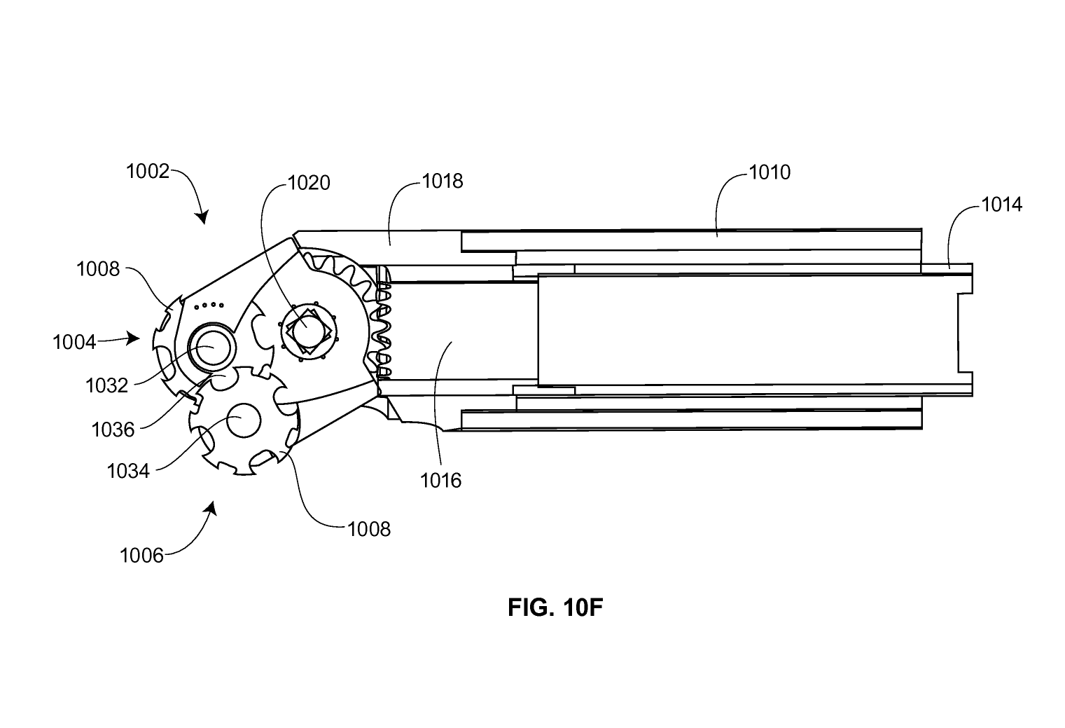

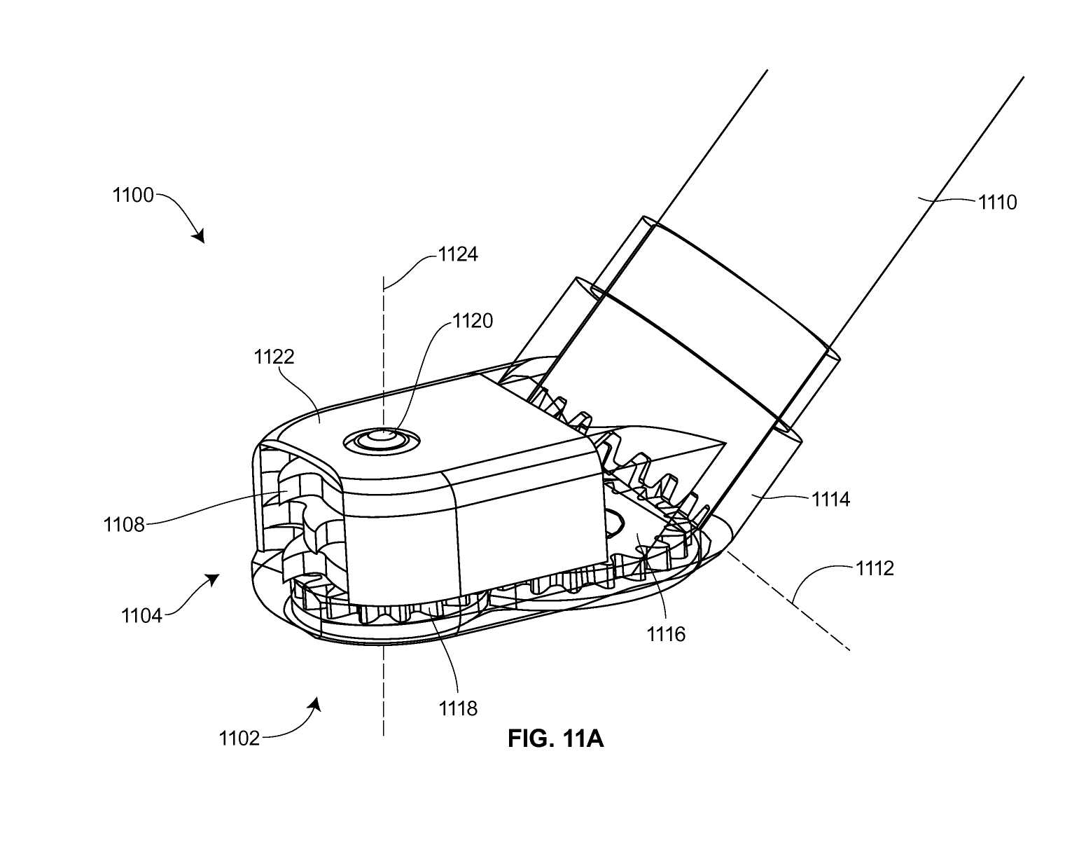

Referring to FIGS. 10A-10F, the distal end of an exemplary embodiment of an angled tissue shredder 1000 is shown. Tissue shredder 1000 is constructed and operates in a manner similar to cutter head assembly 5332 shown in FIGS. 6A-6C, but with rotor housing assembly 1002 turned 30 degrees to one side of the distal end of tissue shredder 1000. This arrangement permits a surgeon to more easily access target tissue located in particularly difficult to reach locations. In this embodiment, rotor housing assembly 1002 includes two rotatable members 1004, 1006. Rotatable member 1004 has two multi-directional cutter blades 1008, and rotatable member 1006 has three multi-directional cutter blades 1008. The rotatable members 1004 and 1006 are further examples of the types of cutter arrangements depicted in FIGS. 8G, 8K and 8M. More specifically, the rotational axes of rotatable members 1004 and 1006 are each offset from and perpendicular to a longitudinal axis of shaft 1010 and lie in a different plane from the longitudinal axis, as also depicted in FIG. 8G. In a variation of device 1000 (not shown), the rotational axes of rotatable members 1004 and 1006 may be pivoted about an articulation axis 1012 that is parallel to the rotational axes, as also depicted in FIG. 8K and subsequently described in more detail. Additionally, a distal portion of a central longitudinal axis of shaft 1010 and a more proximal portion of the central longitudinal axis lie in a common plane that is generally perpendicular to the rotational axis of rotatable members 1004 and 1006, as also depicted in FIG. 8M.

As best seen in FIG. 10B, tissue shredder 1000 includes an outer shaft 1010, an inner drive tube 1014, a gear tube 1016 (which may be integrally formed on the distal end of inner drive tube 1014), lug 1018, aligner pin 1020, aligner cap 1022, rotor housing assembly 1002, and drive gear 1024. Outer shaft 1010 is rigidly affixed to the proximal end of lug 1018. Inner drive tube 1014 and gear to 1016 are concentrically mounted within and rotate with respect to outer shaft 1010 for driving rotatable members 1004 and 1006. Rotor housing assembly 1002 is received within a horizontal slot through lug 1018. Rotor housing assembly 1002 is accurately located within lug 1018 and held in place by aligner pin 1020 and aligner cap 1022, which may be welded together when assembled. Drive gear 1024 is located partially within rotor housing assembly 1002 and receives aligner pin 1020 therethrough during assembly.

As best seen in FIGS. 10C and 10D, gear tube 1016 engages with drive gear 1024 to convert the horizontal axis rotation of gear tube 1016 to the vertical axis rotation of drive gear 1024. Because aligner pin 1020 is rotationally coupled with drive gear 1024, aligner pin 1020 and aligner 1022 attached thereto also rotate about a vertical axis with drive gear 1024.

As shown in FIG. 10E, aligner pin 1020 drives a third gear 1026 located above drive gear 1024 on aligner pin 1020. Third gear 1026 drives a fourth gear 1028, which in turn drives a fifth gear 1030. Through a first axle 1032, fourth gear 1028 drives first rotatable member 1004. Similarly, through a second axle 1034, fifth gear 1030 drives second rotatable member 1006.

As shown in FIG. 10F, cutting blades 1008 of the first and second rotatable members 1004 and 1006 overlap to sheer tissue therebetween. Gap 1036 is provided between interdigitated blades 1008 to permit the sheared tissue pieces to pass through rotatable members 1004 and 1006 and into the interior of rotor housing assembly 1002 for removal through inner drive tube 1014.

In the previously mentioned variation of device 1000, the rotational axes of rotatable members 1004 and 1006 may be pivoted about an articulation axis 1012 that is parallel to the rotational axes. To accommodate such pivoting, one or more tension and/or compression bearing members, such as two pull wires (not shown) may be incorporated into the device. The distal ends of the pull wires may be pivotable affixed to opposite sides of the rotor housing assembly 1002. The proximal ends of the pull wires may be affixed to an articulation lever located at the proximal end of the instrument. By pivoting the articulation lever, the angular orientation of the rotor housing assembly 1002 may be changed during a surgical procedure. By locking and/or by having detent positions of the articulation lever, the angular orientation of the rotor housing assembly 1002 may be locked in place. Bellows, telescoping sections, or other means may be employed to form a seal between rotor housing assembly 1002 and lug 1018 over the range of angular orientations.

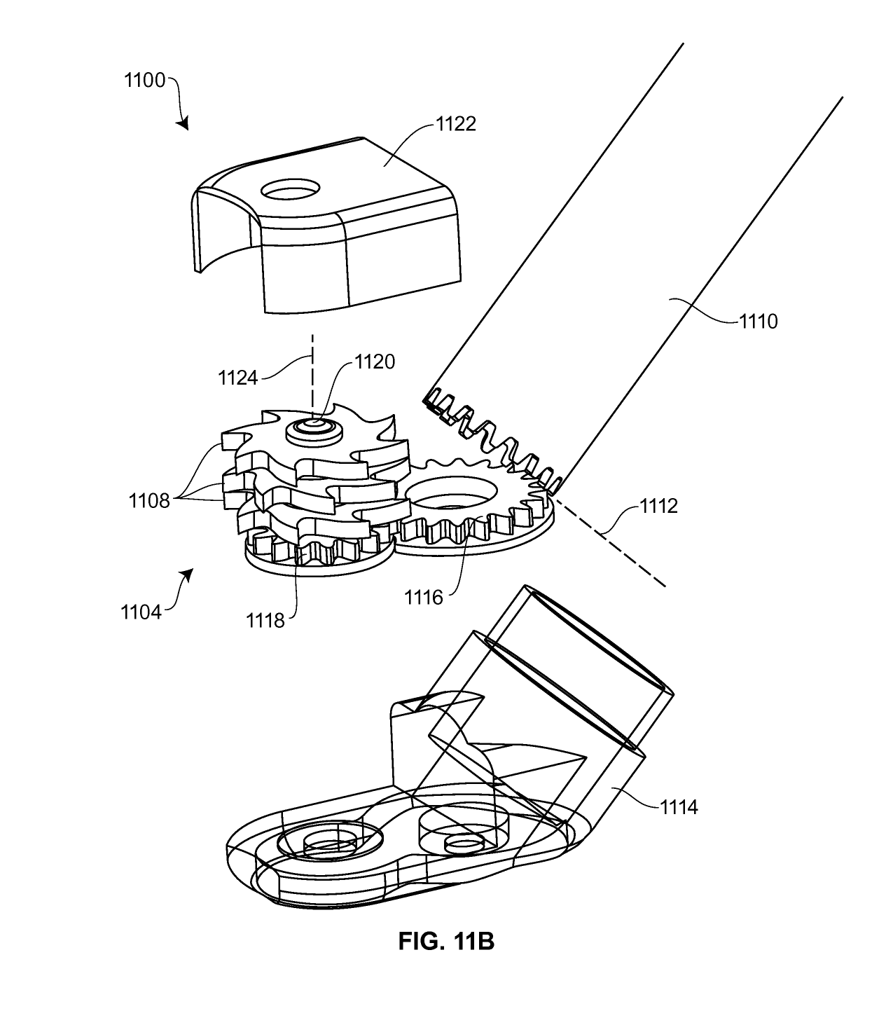

Referring to FIGS. 11A-11B, the distal end of an exemplary embodiment of an angled tissue shredder 1100 is shown. This embodiment permits a surgeon to more easily access target tissue located in particularly difficult to reach locations. In this embodiment, rotor housing assembly 1102 includes a single rotatable member 1104. Rotatable member 1104 has three cutter blades 1108. The rotatable member 1104 is a further example of the type of cutter arrangements depicted in FIGS. 8F, 8I and 80. More specifically, the rotational axis of rotatable member 1104 is offset from a longitudinal axis of shaft 1110, lies in a common plane with the longitudinal axis, and forms an angle therewith of between 0 and 90°, as also depicted in FIG. 8G. In a variation of device 1100 (not shown), the rotational axis of rotatable member 1104 may be pivoted about an articulation axis 1112 that is perpendicular to the rotational axis, as also depicted in FIG. 8I. Additionally, a distal portion of a central longitudinal axis of shaft 1110 and a more proximal portion of the central longitudinal axis lie in a common plane that is coincident with the rotational axis of rotatable member 1104, as also depicted in FIG. 8O.

Similar to previously described embodiments, tissue shredder 1100 includes an inner drive tube 1110 with a crown gear formed on its distal end. Inner drive tube 1110 is rotatably received within the proximal end of lug 1114. A stationary outer tube (not shown) is rigidly affixed to the proximal end of lug 1114. The crown gear of inner drive tube 1110 engages at an angle with idler gear 1116. Idler gear 1116 drives right angle gear 1118, which in turn drives rotatable member 1104 through pin 1120, as with previously described embodiments. Cover 1122 is affixed to lug 1114 to cover all but the distal most portion of rotor 1104.

In operation, teeth on the periphery of blades 1108 engage with tissue distally located from tissue shredder 1100 and draw it inward towards cover 1122, where it is sheared between blades 1108 and cover 1122. The sheared tissue pieces are then drawn into rotor housing assembly 1102 and up into inner drive tube 1110.

In this particular embodiment, axis of rotation 1124 of rotatable member 1104 forms an angle of 45° with inner drive tube 1110. In other embodiments (not shown), angles of between 0 and 90° may be utilized. As with the previous embodiments, device 1100 may be modified such that its angle may be adjusted by a surgeon during use. This articulation may be enabled by pivoting the blade housing with its center located between the meshing of the crown gear at the distal end of inner drive tube 1110 and the flat gear 1116. Cable or pull wires may be used to actuate the angle of the cutter head.

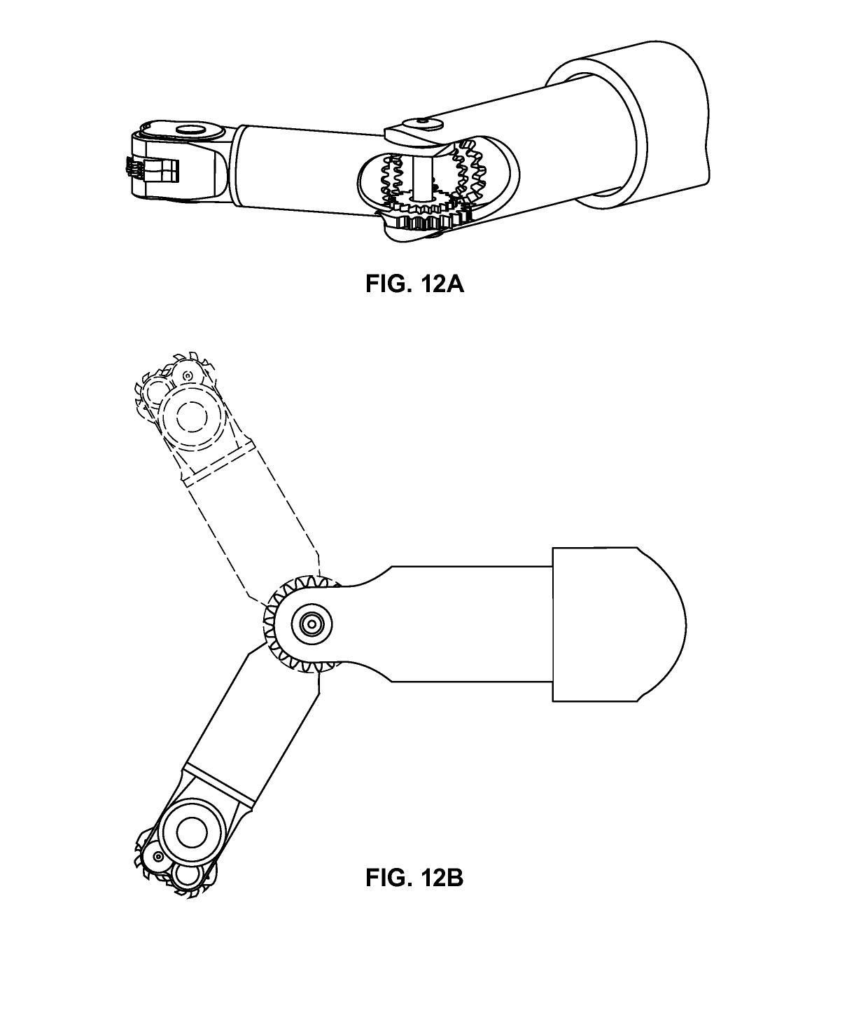

Referring to FIGS. 12A and 12B, an articulating tissue debrider tool 1200 is shown. This embodiment exemplifies a gear drive arrangement that may be used with or may be modified to be used with the previously described embodiments. The distal tip of tool 1200 has a distal housing or lug configured with a tissue cutter assembly. An elongate member is coupled to the distal housing and configured to introduce the distal housing to a target tissue site of a subject, as with previously described embodiments. The elongate member comprises a proximal portion having a first central axis therethrough, and a distal portion having a second central axis therethrough. A joint mechanism is provided between the distal end of the proximal portion and a proximal end of the distal portion. The joint mechanism is configured to allow the distal portion to articulate with respect to the proximal portion, such that the first central axis is non-collinear with the second central axis.

The distal portion of the elongate member includes a distal outer tube and a distal inner drive tube rotatably mounted within the distal outer tube. The distal inner drive tube includes a crown gear at its distal end (not shown) to drive the tissue cutter assembly in a manner similar to previously described embodiments. The distal inner drive tube also includes a crown gear at its proximal end. The crown gear is configured to mesh with a first spur gear of the joint mechanism. The first spur gear is rotatably mounted on a spindle.

The proximal portion of the elongate member includes a proximal outer tube, a proximal inner articulation tube rotatably mounted within the proximal outer tube, and a proximal inner drive tube rotatably mounted within the proximal inner articulation tube. The proximal inner drive tube includes a crown gear at its distal end. The crown gear is configured to mesh with the first spur gear of the joint mechanism. With this arrangement, the proximal inner drive tube may be driven by a motor (not shown) located at the proximal end of device 1200, as with previously described embodiments. The proximal inner drive tube then drives the first spur gear, which in turn drives the distal inner drive tube in an opposite direction from that of the proximal inner drive tube. The distal inner drive tube then rotatably drives the tissue cutter assembly as previously described.

The spindle pivotably interconnects the proximal end of the distal outer tube with the distal end of the proximal outer tube, allowing the two outer tubes to pivot with respect to one another. The proximal and distal inner drive tubes and the first is arranged such that it is able to continually drive the tissue cutter assembly regardless of the orientation the distal outer tube relative to the proximal outer tube. A gear segment is provided at the proximal end of the distal outer tube. The proximal inner articulation tube includes a crown gear at its distal end that is configured to mesh with the gear segment of the distal outer tube. Rotating the proximal end (not shown) of the proximal inner articulation tube, such as with a knob or other control, causes the crown gear at the distal end of the proximal inner articulation tube to pivot the distal portion of the elongate member relative to the proximal portion. FIG. 12B shows the distal portion of the elongate member in a first articulated position, shown with solid lines, and in a second articulated position, shown with phantom lines. The articulation capabilities of the joint mechanism allow device 1200 to approach difficult to reach target tissues from different angles.

The joint mechanism may be provided with a flexible sheath, bellows or other covering (not shown) over the joint to prevent the mechanism from damaging adjacent tissue and to seal irrigation fluid that may be flowing distally and/or proximally through the joint. In some embodiments, irrigation fluid is provided externally adjacent to the tissue cutter assembly. Suction is provided at the proximal end of the proximal inner drive tube to draw the irrigation fluid through the tissue cutter assembly and up through the distal and proximal inner drive tubes, thereby transporting cut tissue debris proximally through the elongate member. In other embodiments, irrigation fluid may be provided distally through channels and/or tubing through the elongate member. In still other embodiments, irrigation fluid may be provided distally through the center of the proximal and distal inner drive tubes.

While exemplary embodiments have been shown having teeth on opposing rotatable members that rotate in sync with one another, in other embodiments the teeth may be arranged so that they are out of sync with one another. In other words, a tooth from one blade may shear tissue with a portion of an opposing blade where there is no tooth, and vice versa. In some embodiments, the rotations of the first and the second rotatable members are configured to alternately rotate in and out of phase with one another. This may be accomplished, for example, by independently driving the rotatable members with separate motors and/or drive trains, by driving two similar rotatable members at different speeds, or driving two dissimilar rotatable members at the same speed.

In some embodiments the first and the second rotatable members are configured to periodically reverse direction of rotation during tissue cutting. This may be done to ensure the tissue cutting head does not clog, to disengage the cutting head from the target tissue, or to engage a different portion of the target tissue, for example. Cutting teeth may be provided that cut equally well in both directions, or are optimized for cutting in a single direction. The rotations of the first and the second rotatable members may be configured to reverse direction at least once per second. In some embodiments the device is configured to provide a dwell time of at least about 50 milliseconds when the first and the second rotatable members reverse direction.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be defined by the claims presented hereafter.

What is claimed is:

1. A medical device for removing tissue from a subject, comprising:
distal housing configured with at least one tissue engaging opening;
an elongate member coupled to the distal housing and configured to introduce the distal housing to a target tissue site of the subject, the elongate member having a central longitudinal axis;
a first rotatable member located at least partially within the distal housing and configured to rotate about a singular first axis, the first rotatable member comprising a first cutting blade, the first blade having a first side and a second side opposite the first side;
a first tissue shearing surface located and configured to cooperate with the first side of the first blade to shear tissue therebetween; and
a second tissue shearing surface located and configured to cooperate with the second side of the first blade to shear tissue therebetween, the first rotatable member configured to engage tissue from the target tissue site, rotate towards the first and second tissue shearing surfaces and inwardly to direct tissue from the target tissue site through the tissue engaging opening and into an interior portion of the distal housing.

2. The medical device of claim 1, wherein the first cutting blade comprises a disc-shaped portion having a series of teeth along an outer circumference of the blade.

3. The medical device of claim 2, wherein the disc-shaped portion is perpendicular to the singular first axis.

4. The medical device of claim 1, wherein at least one of the first and second tissue shearing surfaces is formed by a fixed portion of the distal housing.

5. The medical device of claim 1, wherein the first axis of the first rotatable member is coincident with the longitudinal axis of the elongate member.

6. The medical device of claim 1, wherein the first axis of the first rotatable member intersects the longitudinal axis of the elongate member and is perpendicular therewith.

7. The medical device of claim 1, wherein the first axis of the first rotatable member intersects the longitudinal axis of the elongate member and forms an angle therewith of between 0 and 90 degrees.

8. The medical device of claim 1, wherein the first axis of the first rotatable member is offset from and parallel to the longitudinal axis of the elongate member and lies in a common plane therewith.

9. The medical device of claim 1, wherein the first axis of the first rotatable member is offset from and perpendicular to the longitudinal axis of the elongate member and lies in a common plane therewith.

10. The medical device of claim 1, wherein the first axis of the first rotatable member is offset from the longitudinal axis of the elongate member, lies in a common plane and forms an angle therewith of between 0 and 90 degrees.

11. The medical device of claim 1, wherein the first axis of the first rotatable member is offset from and perpendicular to the longitudinal axis of the elongate member and lies in a different plane.

12. The medical device of claim 11, wherein at least one of the first and second tissue shearing surfaces is formed by a second rotatable member located at least partially within the distal housing and configured to rotate about a singular second axis parallel to and offset from the first axis, the second rotatable member configured to rotate in a direction opposite of a direction of rotation of the first rotatable member, the second rotatable member comprising a second disc-shaped blade having a series of teeth along an outer circumference of the blade.

13. The medical device of claim 12, wherein the second rotatable member comprises a third disc-shaped blade having a series of teeth along an outer circumference of the blade, wherein the three blades are positioned such that they are interdigitated with one another.

14. The medical device of claim 1, wherein the first axis of the first rotatable member is offset from the longitudinal axis of the elongate member, lies in a different plane and forms an angle therewith of between 0 and 90 degrees.

15. The medical device of claim 14, wherein at least one of the first and second tissue shearing surfaces is formed by a second rotatable member located at least partially within the distal housing and configured to rotate about a singular second axis parallel to and offset from the first axis, the second rotatable member configured to rotate in a direction opposite of a direction of rotation of the first rotatable member, the second rotatable member comprising a second disc-shaped blade having a series of teeth along an outer circumference of the blade.

16. The medical device of claim 15, wherein the second rotatable member comprises a third disc-shaped blade having a series of teeth along an outer circumference of the blade, wherein the three blades are positioned such that they are interdigitated with one another.

17. The medical device of claim 1, wherein the first axis of the first rotatable member is perpendicular to the longitudinal axis of the elongate member and is configured to articulate with respect thereto.

18. The medical device of claim 17, wherein the first axis pivots about an articulation axis that is parallel thereto.

19. The medical device of claim 17, wherein the first axis pivots about an articulation axis that is perpendicular thereto.

20. The medical device of claim 1, wherein the elongate member comprises a distal portion that is oriented at an angle with respect to a more proximal portion of the elongate member such that the central longitudinal axis has an inflection point between the distal portion and more proximal portion.

21. The medical device of claim 20, wherein a distal portion of the central longitudinal axis and a more proximal portion of the central longitudinal axis lie in a common plane that is coincident with or generally parallel to the first axis of the first rotatable member.

22. The medical device of claim 20, wherein a distal portion of the central longitudinal axis and a more proximal portion of the central longitudinal axis lie in a common plane that is generally perpendicular to the first axis of the first rotatable member.

23. The medical device of claim 1, wherein the elongate member comprises a generally rigid, curved distal portion and a generally straight more proximal portion.

24. The medical device of claim 23, wherein a curved, distal portion of the central longitudinal axis lies in a plane that is coincident with or generally parallel to the first axis of the first rotatable member.

25. The medical device of claim 23, wherein a curved distal portion of the central longitudinal axis lies in a plane that is generally perpendicular to the first axis of the first rotatable member.
